SEQUENCE LISTING (25 sequences)

SEQ ID NO: 1 - protein, 117 aa, synthetic construct [sequence omitted]

SEQ ID NO: 2 - protein, 107 aa, synthetic construct [sequence omitted]

SEQ ID NO: 3 - protein, 8 aa, synthetic construct
  REGION 1..8: note = Synthetic
GFTFSNFG

SEQ ID NO: 4 - protein, 8 aa, synthetic construct
  REGION 1..8: note = Synthetic
ISGGGRDT

SEQ ID NO: 5 - protein, 10 aa, synthetic construct
  REGION 1..10: note = Synthetic
VKWGNIYFDY

SEQ ID NO: 6 - protein, 6 aa, synthetic construct
  REGION 1..6: note = Synthetic
LSINTF

SEQ ID NO: 7 - skipped

SEQ ID NO: 8 - protein, 9 aa, synthetic construct
  REGION 1..9: note = Synthetic
QQSSNTPFT

SEQ ID NO: 9 - protein, 444 aa, synthetic construct
  REGION 1..444: note = Synthetic
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
  REGION 1..214: note = Synthetic
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 120 aa, synthetic construct
  REGION 1..120: note = Synthetic
EVQLVESGGGVVRPGGSLRLSCAASGFTFDDYGMTWVRQAPGRGLEWVSGIHWHGKRTGYADSVKGRFTISRDNAKKSLYLQMNSLKGEDTALYHCVRGGMSTGDWFDPWGQGTLVIVSS

SEQ ID NO: 12 - protein, 108 aa, synthetic construct
  REGION 1..108: note = Synthetic
DIQMTQSPSSLSASLGDRVTITCRASQSINSYLNWYQQKPGKAPKLLIYVASSLQSGVPSRFSGSGSGTEFTLTISNLQPEDFATYYCQQSYSTPPITFGQGTRLEIK

SEQ ID NO: 13 - protein, 116 aa, synthetic construct
  REGION 1..116: note = Synthetic
EVQLVESGGGLVQPGGSLRLSCAASGFTFSNYEMSWVRQAPGKGLEWVSSIRTSGTTKYYADSMKGRFTISRDNAKNSLYLQMNSLRAEDTAVYYCAGGGTFLHYWGQGTLVTVSS

SEQ ID NO: 14 - protein, 108 aa, synthetic construct
  REGION 1..108: note = Synthetic
DIQMTQSPSSVSASVGDRVTITCRASQGIASYLAWYQQKPGKAPKLLIYAASSLQTGVPSRFSGSGYGTDFTLTISSLQPEDFATYYCQQAKSFPMYTFGQGTKLEIK

SEQ ID NO: 15 - protein, 8 aa, synthetic construct
  REGION 1..8: note = Synthetic
GFTFSNYE

SEQ ID NO: 16 - protein, 8 aa, synthetic construct
  REGION 1..8: note = Synthetic
IRTSGTTK

SEQ ID NO: 17 - protein, 9 aa, synthetic construct
  REGION 1..9: note = Synthetic
AGGGTFLHY

SEQ ID NO: 18 - protein, 6 aa, synthetic construct
  REGION 1..6: note = Synthetic
QGIASY

SEQ ID NO: 19 - skipped

SEQ ID NO: 20 - protein, 10 aa, synthetic construct
  REGION 1..10: note = Synthetic
QQAKSFPMYT

SEQ ID NO: 21 - protein, 446 aa, synthetic construct
  REGION 1..446: note = Synthetic
EVQLVESGGGLVQPGGSLRLSCAASGFTFSNYEMSWVRQAPGKGLEWVSSIRTSGTTKYYADSMKGRFTISRDNAKNSLYLQMNSLRAEDTAVYYCAGGGTFLHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 215 aa, synthetic construct
  REGION 1..215: note = Synthetic
DIQMTQSPSSVSASVGDRVTITCRASQGIASYLAWYQQKPGKAPKLLIYAASSLQTGVPSRFSGSGYGTDFTLTISSLQPEDFATYYCQQAKSFPMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 182 aa, Mus musculus
MNNRWILHAAFLLCFSTTALSINYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQN

SEQ ID NO: 24 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 25 - protein, 643 aa, Homo sapiens
MEAVETGERPTFGAWDYGVFALMLLVSTGIGLWVGLARGGQRSAEDFFTGGRRLAALPVGLSLSASFMSAVQVLGVPSEAYRYGLKFLWMCLGQLLNSVLTALLFMPVFYRLGLTSTYEYLEMRFSRAVRLCGTLQYIVATMLYTGIVIYAPALILNQVTGLDIWASLLSTGIICTFYTAVGGMKAVVWTDVFQVVVMLSGFWVVLARGVMLVGGPRQVLTLAQNHSRINLMDFNPDPRSRYTFWTFVVGGTLVWLSMYGVNQAQVQRYVACRTEKQAKLALLINQVGLFLIVSSAACCGIVMFVFYTDCDPLLLGRISAPDQYMPLLVLDIFEDLPGVPGLFLACAYSGTLSTASTSINAMAAVTVEDLIKPRLRSLAPRKLVIISKGLSLIYGSACLTVAALSSLLGGGVLQGSFTVMGVISGPLLGAFILGMFLPACNTPGVLAGLGAGLALSLWVALGATLYPPSEQTMRVLPSSAARCVALSVNASGLLDPALLPANDSSRAPSSGMDASRPALADSFYAISYLYYGAPGTLTTVLCGALISCLTGPTKRSTLAPGLLWWDLARQTASVAPKEEVAILDDNLVKGPEELPTGNKKPPGFLPTNEDRLFFLGQKELEGAGSWTPCVGHDGGRDQQETNL

(19) United States
(12) Patent Application Publication
(10) Pub. No.: US 2023/0139492 A1
(43) Pub. Date: May 4, 2023

(54) COMBINATION OF CHECKPOINT INHIBITORS AND AN ONCOLYTIC VIRUS FOR TREATING CANCER

(71) Applicant: Regeneron Pharmaceuticals, Inc., Tarrytown, NY (US)

(72) Inventors: Saida Dadi-Mehmetaj, Ardsley, NY (US); Markus Mohrs, Pleasantville, NY (US); Gavin Thurston, Briarcliff Manor, NY (US)

(21) Appl. No.: 17/813,100

(22) Filed: Jul. 18, 2022

Related U.S. Application Data
(60) Provisional application No. 63/365,030, filed on May 20, 2022.
(60) Provisional application No. 63/223,281, filed on Jul. 19, 2021.

(51) Int. Cl. C07K 16/28; C12N 7/00; A61P 35/00

(52) U.S. Cl. CPC C07K 16/2896; C07K 16/2818; C12N 7/00; A61P 35/00; C12N 2760/20232; C07K 2317/565

(57) ABSTRACT

This disclosure relates to novel triple combination therapies of an oncolytic virus, a PD-1 pathway inhibitor, and a CTLA4 inhibitor for treating or inhibiting the growth of a tumor in a patient with cancer.

### CROSS-REFERENCE TO RELATED APPLICATIONS

This application claims the benefit of priority to U.S. Provisional Patent Application No. 63/223,281 filed Jul. 19, 2021, and U.S. Provisional Patent Application No. 63/365,030 filed May 20, 2022, the disclosures of all of which are hereby incorporated by reference herein in their entireties.

### FIELD

The present disclosure relates generally to combination therapies for cancer treatment with oncolytic viruses and checkpoint inhibitors such as programmed death 1 (PD-1) pathway inhibitors and cytotoxic T-lymphocyte antigen-4 (CTLA4) inhibitors.

### BACKGROUND

Until recently, cancer immunotherapy had focused substantial effort on approaches that enhance anti-tumor immune responses by adoptive-transfer of activated effector cells, immunization against relevant antigens, or providing non-specific immune stimulatory agents such as cytokines. In the past decade, however, intensive efforts to develop specific immune checkpoint pathway inhibitors have begun to provide new immunotherapeutic approaches for treating cancer, including the development of anti-PD-1 antibodies and anti-CTLA4 antibodies.

PD-1 (also known as CD279) plays an important role in autoimmunity, immunity against infection, and anti-tumor immunity. Blocking PD-1 with antagonists, including monoclonal antibodies, has been studied in treatments of cancer and chronic viral infections. Blockade of PD-1 is also an effective and well-tolerated approach to stimulating the immune response, and has achieved therapeutic advantage against various human cancers, including melanoma, renal cell cancer (RCC), and non-small cell lung cancer (NSCLC). (Sheridan 2012, Nat. Biotechnol., 30:729-730; Postow et al., 2015, J Clin Oncol, 33:1974-1982).

CTLA4 (also known as CD152) is a type I transmembrane T cell inhibitory checkpoint receptor expressed on conventional and regulatory T cells. CTLA4 negatively regulates T cell activation by outcompeting the stimulatory receptor CD28 from binding to its shared natural ligands, B7-1 (CD80) and B7-2 (CD86).

Initial T-cell activation is achieved by stimulating T-cell receptors (TCR) that recognize specific peptides presented by major histocompatibility complex class I or II (MHCI or MHCII) proteins on antigen-presenting cells (APC) (Goldrath et al. 1999, Nature 402: 255-262). An activated TCR complex in turn initiates a cascade of signaling events driven by promoters regulating the expression of various transcription factors such as activator-protein 1 (AP-1), Nuclear Factor of Activated T-cells (NFAT) or Nuclear factor kappa-light-chain-enhancer of activated B cells (NF-kappa-B). The T-cell response is then further modulated via engagement of co-stimulatory or co-inhibitory receptors expressed either constitutively or inducibly on T-cells such as CD28, CTLA4, PD-1, Lymphocyte-Activation Gene 3 (LAG-3) or other molecules (Sharpe et al. 2002, Nat. Rev. Immunol. 2: 116-126).

Oncolytic viruses also hold promise for the treatment of cancer. These viruses infect, specifically replicate in, and kill malignant cells leaving normal tissues unaffected. Several oncolytic viruses have reached advanced stages of clinical evaluation for the treatment of a variety of neoplasms. However, immune suppression by tumors and premature clearance of the virus often result in only weak tumor-specific immune responses, limiting the potential of these viruses as a cancer therapeutic.

Accordingly, there exists a strong need for more effective therapies for cancer treatment, including, as disclosed herein, combination therapies comprising oncolytic viruses and checkpoint inhibitors such as PD-1 pathway inhibitors and CTLA4 inhibitors.

### SUMMARY

In one aspect, the disclosed technology relates to a method of treating or inhibiting the growth of a tumor, including: (a) selecting a patient with a cancer; and (b) administering to the patient in need thereof: (i) a therapeutically effective amount of an oncolytic virus in combination with (ii) a therapeutically effective amount of a programmed death 1 (PD-1) pathway inhibitor, and (iii) a therapeutically effective amount of a cytotoxic T-lymphocyte antigen-4 (CTLA4) inhibitor. In some embodiments, the oncolytic virus includes an oncolytic vesiculovirus. In some embodiments, the oncolytic vesiculovirus includes an oncolytic vesicular stomatitis virus (VSV). In some embodiments, the VSV includes a recombinant VSV. In some embodiments, the recombinant VSV includes one or more mutations, such as an M51R substitution. In some embodiments, the recombinant VSV expresses a cytokine. In some embodiments, the recombinant VSV contains a nucleic acid sequence encoding an immunostimulatory molecule such as a cytokine.

In some embodiments, the cytokine includes an interferon-beta (IFNb), such as a human or mouse IFNb or a variant thereof. In some embodiments, a nucleic acid sequence encoding the IFNb is positioned between M and G vial genes.

In some embodiments, the recombinant VSV further expresses a sodium/iodide symporter (NIS). In some embodiments, the recombinant VSV further contains a nucleic acid sequence encoding for a sodium/iodide symporter (NIS) or a variant thereof. In some embodiments, a nucleic acid sequence encoding the NIS is positioned between G and L viral genes. In some embodiments, the oncolytic virus is Voyager V1. In some embodiments, the oncolytic virus, the PD-1 pathway inhibitor, and the CTLA4 inhibitor are administered concurrently to the patient. In some embodiments, one or more doses of the oncolytic virus are administered sequentially in combination with one or more doses of the PD-1 pathway inhibitor and one or more doses of the CTLA4 inhibitor.

In some embodiments, the oncolytic virus is administered to the patient before or after the PD-1 pathway inhibitor and/or the CTLA4 inhibitor. In some embodiments, the PD-1 pathway inhibitor is administered to the patient before or after the oncolytic virus and/or the CTLA4 inhibitor. In some embodiments, the CTLA4 inhibitor is administered to the patient before or after the oncolytic virus and/or the PD-1 pathway inhibitor. In some embodiments, at least one of the oncolytic virus, the PD-1 pathway inhibitor, or the CTLA4 inhibitor is administered to the patient once a day, once every two days, once every three days, once every four days, once every five days, once every week, once every two weeks, or once every three weeks. In some embodiments, a dose of the oncolytic virus, the PD-1 pathway inhibitor, or the CTLA4 inhibitor is administered to the patient 1 day to 12 weeks after an immediately preceding dose of the oncolytic virus, the PD-1 pathway inhibitor, or the CTLA4 inhibitor, respectively.

In some embodiments, one or more doses of the CTLA4 inhibitor include a single dose of the CTLA4 inhibitor and wherein administration of the single dose of the CTLA4 inhibitor leads to an anti-tumor efficacy comparable to that with a combination therapy including two or more doses of the CTLA4 inhibitor.

In some embodiments, the anti-tumor efficacy is characterized by decrease in mean or average tumor volume, percent survival, numbers of tumor free patients in each treatment group, or a combination thereof. In some embodiments, the oncolytic virus is administered to the patient as one or more doses of 10⁴-10¹⁴ TCID₅₀ (50% Tissue Culture Infectious Dose), 10⁴-10¹² TCID₅₀, 10⁶-10¹² TCID₅₀, 10⁸-10¹⁴ TCID₅₀, 10⁸-10¹² TCID₅₀ or 10¹⁰-10¹² TCID₅₀. In some embodiments, the PD-1 pathway inhibitor is administered to the patient in one or more doses of about 0.1 mg/kg to about 20 mg/kg of body weight of the patient. In some embodiments, the PD-1 pathway inhibitor is administered to the patient in one or more doses of about 1 mg to about 1000 mg. In some embodiments, the CTLA4 inhibitor is administered to the patient in one or more doses of about 0.1 mg/kg to about 15 mg/kg of body weight of the patient. In some embodiments, the CTLA4 inhibitor is administered to the patient in a single dose of about 0.1 mg/kg to about 15 mg/kg of body weight of the patient. In some embodiments, the CTLA4 inhibitor is administered to the patient in one or more doses of about 1 mg to about 600 mg. In some embodiments, the oncolytic virus is administered intratumorally or intravenously to the patient. In some embodiments, the PD-1 pathway inhibitor and the CTLA4 inhibitor are administered intravenously, subcutaneously or intraperitoneally to the patient.

In some embodiments, the cancer is selected from adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, lymphoma, melanoma, meningioma, multiple neuroendocrine type I and type II tumors, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumors, prostate cancer, rectal cancer, renal cancer, respiratory cancer, sarcoma, skin cancer, stomach cancer, testicular cancer, thyroid cancer, tracheal cancer, urogenital cancer, and uterine cancer. In some embodiments, the cancer is resistant to treatment with at least one anti-PD-1 agent or therapy.

In some embodiments, the PD-1 pathway inhibitor includes an anti-PD-1 antibody or antigen-binding fragment thereof, an anti-PD-L1 antibody or antigen-binding fragment thereof, or an anti-PD-L2 antibody or antigen-binding fragment thereof. In some embodiments, the anti-PD-1 antibody is selected from cemiplimab, nivolumab, pembrolizumab, pidilizumab, MED10608, BI 754091, PF-06801591, spartalizumab, camrelizumab, JNJ-63723283, and MCLA-134.

In some embodiments, the anti-PD-1 antibody or antigen-binding fragment thereof includes the heavy chain complementarity determining regions (HCDRs) of a heavy chain variable region (HCVR) including the amino acid sequence of SEQ ID NO: 1 and the light chain complementarity determining regions (LCDRs) of a light chain variable region (LCVR) including the amino acid sequence of SEQ ID NO: 2. In some embodiments, the anti-PD-1 antibody or antigen-binding fragment thereof includes three heavy chain complementarity determining regions (HCDRs) (HCDR1, HCDR2, and HCDR3) including the respective amino acid sequences of SEQ ID NOs: 3, 4, and 5; and three light chain CDRs (LCDR1, LCDR2, and LCDR3) including the respective amino acid sequences of SEQ ID NOs: 6, 7, and 8.

In some embodiments, the anti-PD-1 antibody or antigen-binding fragment thereof includes a heavy chain variable region (HCVR) including the amino acid sequence of SEQ ID NO: 1; and a light chain variable region (LCVR) including the amino acid sequence of SEQ ID NO: 2. In some embodiments, the anti-PD-1 antibody or antigen-binding fragment thereof includes a heavy chain and light chain sequence pair of SEQ ID NOs: 9 and 10.

In some embodiments, the anti-PD-L1 antibody is selected from REGN3504, avelumab, atezolizumab, durvalumab, MDX-1105, LY3300054, FAZ053, STI-1014, CX-072, KN035, and CK-301. In some embodiments, the anti-PD-L1 antibody or antigen-binding fragment thereof includes a heavy chain variable region (HCVR) including the amino acid sequence of SEQ ID NO: 11; and a light chain variable region (LCVR) including the amino acid sequence of SEQ ID NO: 12. In some embodiments, the anti-PD-L1 antibody includes REGN3504.

In some embodiments, the CTLA4 inhibitor includes an anti-CTLA4 antibody or antigen-binding fragment thereof. In some embodiments, the anti-CTLA4 antibody is selected from ipilimumab, tremelimumab, and REGN4659. In some embodiments, the anti-CTLA4 antibody or antigen-binding fragment thereof includes the heavy chain complementarity determining regions (HCDRs) of a heavy chain variable region (HCVR) including the amino acid sequence of SEQ ID NO: 13 and the light chain complementarity determining regions (LCDRs) of a light chain variable region (LCVR) including the amino acid sequence of SEQ ID NO: 14.

In some embodiments, the anti-CTLA4 antibody or antigen-binding fragment thereof includes three heavy chain complementarity determining regions (HCDRs) (HCDR1, HCDR2, and HCDR3) including the respective amino acid sequences of SEQ ID NOs: 15, 16, and 17; and three light chain CDRs (LCDR1, LCDR2, and LCDR3) including the respective amino acid sequences of SEQ ID NOs: 18, 19, and 20. In some embodiments, the anti-CTLA4 antibody or antigen-binding fragment thereof includes a heavy chain variable region (HCVR) including the amino acid sequence of SEQ ID NO: 13; and a light chain variable region (LCVR) including the amino acid sequence of SEQ ID NO: 14. In some embodiments, the anti-CTLA4 antibody or antigen-binding fragment thereof includes a heavy chain and light chain sequence pair of SEQ ID NOs: 21 and 22.

In some embodiments, the treatment produces a therapeutic effect selected from one or more of: delay in tumor growth, reduction in tumor cell number, tumor regression, increase in survival, partial response, and complete response. In some embodiments, the tumor growth is inhibited by at least 50% as compared to an untreated patient. In some embodiments, the tumor growth is inhibited by at least 50% as compared to a patient administered the oncolytic virus, the PD-1 pathway inhibitor, or the CTLA4 inhibitor as monotherapy. In some embodiments, the tumor growth is inhibited by at least 50% as compared to a patient administered any two of the oncolytic virus, the PD-1 pathway inhibitor, and the CTLA4 inhibitor.

In some embodiments, the method further includes administering an additional therapeutic agent or therapy to the patient. In some embodiments, the additional therapeutic agent or therapy is selected from: radiation, surgery, a chemotherapeutic agent, a cancer vaccine, a B7-H3 inhibitor, a B7-H4 inhibitor, a lymphocyte activation gene 3 (LAG3) inhibitor, a T cell immunoglobulin and mucin-domain containing-3 (TIM3) inhibitor, a galectin 9 (GAL9) inhibitor, a V-domain immunoglobulin (Ig)-containing suppressor of T-cell activation (VISTA) inhibitor, a Killer-Cell Immunoglobulin-Like Receptor (KIR) inhibitor, a B and T lymphocyte attenuator (BTLA) inhibitor, a T cell immunoreceptor with Ig and ITIM domains (TIGIT) inhibitor, a CD47 inhibitor, an indoleamine-2,3-dioxygenase (IDO) inhibitor, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an antibody to a tumor-specific antigen, Bacillus Calmette-Guerin vaccine, granulocyte-macrophage colony-stimulating factor (GM-CSF), a cytotoxin, an interleukin 6 receptor (IL-6R) inhibitor, an interleukin 4 receptor (IL-4R) inhibitor, an IL-10 inhibitor, IL-2, IL-7, IL-12, IL-21, IL-15, an antibody-drug conjugate, an anti-inflammatory drug, and combinations thereof.

In another aspect, the disclosed technology relates to a combination of an oncolytic virus, a PD-1 pathway inhibitor, and a CTLA4 inhibitor for use in a method of treating or inhibiting the growth of a tumor, the method including: (a) selecting a patient with a cancer; and (b) administering to the patient in need thereof: (i) a therapeutically effective amount of the oncolytic virus in combination with (ii) a therapeutically effective amount of the PD-1 pathway inhibitor, and (iii) a therapeutically effective amount of the CTLA inhibitor.

In another aspect, the disclosed technology relates to a kit including an oncolytic virus, a PD-1 pathway inhibitor, and a CTLA4 inhibitor, in combination with written instructions for use of a therapeutically effective amount of a combination of the oncolytic virus, the PD-1 pathway inhibitor, and the CTLA4 inhibitor for treating or inhibiting the growth of a tumor of a patient.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 is a graph showing anti-tumor efficacy of the combination treatment with anti-PD-1, anti-CTLA4, and intra-tumor delivery of oncolytic virus VSV-M51R-Fluc in mice bearing 150 mm³ average MC38 tumors as described in Example 1. Average tumor volumes (mm³+/−SEM) in each treatment group at multiple post-tumor implantation time points are shown, with treatment days indicated by arrows, as described in Example 1.

FIG. 2 is a graph showing individual tumor volumes at day 11 after treatment initiation (day 26 after tumor implantation) for each treatment group described in Example 1.

FIG. 3 is a graph showing Kaplan-Meier survival curves for each treatment group described in Example 1.

FIGS. 4A, 4B, 4C, 4D, and 4E are a set of diagrams showing anti-tumor efficacy of the triple combination anti-PD-1, anti-CTLA4, and oncolytic virus VSV-M51R-GFP delivered intra-tumor can be achieved with only one dose of anti-CTLA4 mlgG2a antibody as described in Example 2. FIG. 4A shows average tumor volumes in the PBS treated group at multiple post-tumor implantation time points. FIG. 4B shows average tumor volumes in the PBS, anti-PD-1 antibody, and anti-CTLA4 antibody (4 doses) treated group at multiple post-tumor implantation time points. FIG. 4C shows average tumor volumes in the VSV, anti-PD-1 antibody, and anti-CTLA4 antibody (1 dose) treated group at multiple post-tumor implantation time points. FIG. 4D shows average tumor volumes in the VSV, anti-PD-1 antibody, and anti-CTLA4 antibody (2 doses) treated group at multiple post-tumor implantation time points. FIG. 4E shows average tumor volumes in the VSV IT, anti-PD-1 antibody, and anti-CTLA4 antibody (4 doses) treated group at multiple post-tumor implantation time points. Treatment days are indicated by arrows. TF: tumor free.

FIG. 5 is a graph showing Kaplan-Meier survival curves for each treatment group described in Example 2.

FIG. 6 is a graph showing that anti-tumor efficacy of the triple combination anti-PD-1, anti-CTLA4, and oncolytic virus VSV-M51R-GFP can be achieved with either intra-tumor or intravenous delivery of the virus as described in Example 3. Average tumor volumes (mm³+/−SEM) in each treatment group at multiple post-tumor implantation time points are shown. Treatments were administered as described in Table 5 and Example 3.

FIG. 7 is a graph showing Kaplan-Meier survival curves for each treatment group described in Example 3.

FIG. 8 is a graph showing anti-tumor efficacy of the combination treatment with anti-PD-1, anti-CTLA4, and intravenous delivery of oncolytic virus VSV-mlFNb-NIS in mice bearing 150 mm³ average MC38 tumors as described in Example 4. Average tumor volumes (mm³+/−SEM) in each treatment group at multiple post-tumor implantation time points are shown. Treatments were administered as described in Table 7 and Example 4.

FIG. 9 is a graph showing individual tumor volumes at day 10 after treatment initiation for each treatment group described in Example 4. Statistical significance was determined by one-way ANOVA with Dunnett's multiple comparisons post-test (** p<0.01, **** p<0.0001).

FIG. 10 is a graph showing Kaplan-Meier survival curves for each treatment group described in Example 4.

FIG. 11 is a graph showing average tumor volumes (mm³+/−SEM) in each treatment group described in Example 5 at multiple post-tumor implantation time points, with treatment days indicated by arrows.

FIG. 12 is a graph showing individual tumor volumes at day 29 after treatment initiation for each treatment group described in Example 5.

FIG. 13 is a graph showing Kaplan-Meier survival curves for each treatment group described in Example 5.

FIG. 14 is a graph showing average tumor volumes (mm³+/−SEM) in each treatment group described in Example 6 at multiple post-tumor implantation time points, with treatment days indicated by arrows.

FIG. 15 is a graph showing individual tumor volumes at day 22 after post-tumor implantation (10 days post treatment initiation) for each treatment group described in Example 6.

FIG. 16 is a graph showing Kaplan-Meier survival curves for each treatment group described in Example 6.

FIG. 17 is a graph showing average tumor volumes (mm³+/−SEM) in each treatment group described in Example 7 at multiple post-tumor implantation time points, with treatment days indicated by arrows.

FIG. 18 is a graph showing average tumor volumes (mm³+/−SEM) in each treatment group described in Example 8 at multiple post-tumor implantation time points, with treatment days indicated by arrows.

FIG. 19 is a graph showing average tumor volumes (mm³+/−SEM) in each treatment group described in Example 9 at multiple post-tumor implantation time points.

FIG. 20 is a graph showing average spot forming units (SFU) of IFNg released by CD8 TILs harvested from tumors and re-exposed overnight to the indicated tumor antigen or VSV-NP in each treatment group described in Example 10 at day 17 after receiving VSV at day 12 along with two doses of anti-PD-1 and a-CTLA4 at day 12 and 14. DMSO and PMA/Ionomycin serve as negative and positive controls respectively multiple post-tumor implantation time points, with treatment days indicated by arrows.

### DETAILED DESCRIPTION

This disclosure is based, at least in part, on an unexpected discovery that novel triple combination therapies of an oncolytic virus, a programmed death 1 (PD-1) pathway inhibitor, and a cytotoxic T-lymphocyte antigen-4 (CTLA4) inhibitor exhibit synergistic activity in inhibiting tumor growth than any of the monotherapies or dual combination therapies of the oncolytic virus, the PD-1 pathway inhibitor, and the CTLA4 inhibitor. As demonstrated herein, the disclosed triple combination therapy comprising one dose of the CTLA4 inhibitor administered achieved an anti-tumor efficacy comparable to a combination therapy comprising 2, 3, 4 or more doses of the CTLA4 inhibitor. In addition, intravenous administration of the oncolytic virus is at least as efficacious as intratumoral administration of the virus. Thus, the triple combination therapy as disclosed herein represents a surprisingly effective therapy for cancer treatment with a reduced risk of treatment-related toxicity.

Accordingly, in one aspect, this disclosure provides a method of treating or inhibiting the growth of a tumor, including: (a) selecting a patient with a cancer; and (b) administering to the patient in need thereof: (i) a therapeutically effective amount of an oncolytic virus in combination with (ii) a therapeutically effective amount of a PD-1 pathway inhibitor (e.g., an anti-PD-1, anti-PD-L1, or anti-PD-L2 antibody, or antigen-binding fragment thereof) and (iii) a therapeutically effective amount of a CTLA4 inhibitor (e.g., an anti-CTLA4 antibody or antigen-binding fragment thereof).

As used herein, the term “patient” may be interchangeably used with the term “subject.” The expression “a subject in need thereof” means a human or non-human mammal that exhibits one or more symptoms or indications of cancer and/or who has been diagnosed with cancer. In some embodiments, a human subject may be diagnosed with a primary or a metastatic tumor and/or with one or more symptoms or indications including, but not limited to, enlarged lymph node(s), swollen abdomen, chest pain/pressure, unexplained weight loss, fever, night sweats, persistent fatigue, loss of appetite, enlargement of spleen, itching. The expression includes patients who have received one or more cycles of chemotherapy with toxic side effects. In some embodiments, the expression “a subject in need thereof” includes patients with cancer that has been treated but which has subsequently relapsed or metastasized. For example, patients that may have received treatment with one or more anti-cancer agents leading to tumor regression; however, subsequently have relapsed with cancer resistant to the one or more anti-cancer agents (e.g., chemotherapy-resistant cancer) are treated with the methods of the present disclosure.

As used herein, the terms “treating,” “treat,” or the like mean to alleviate or reduce the severity of at least one symptom or indication, to eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, to prevent or inhibit metastasis, to inhibit metastatic tumor growth, to eliminate the need for radiation or surgery, and/or to increase duration of survival of the subject.

In many embodiments, the terms “tumor,” “lesion,” “tumor lesion,” “cancer,” and “malignancy” are used interchangeably and refer to one or more cancerous growths. In some embodiments, the cancer is selected from adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, lymphoma, melanoma, meningioma, multiple neuroendocrine type I and type II tumors, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumors, prostate cancer, rectal cancer, renal cancer, respiratory cancer, sarcoma, skin cancer, stomach cancer, testicular cancer, thyroid cancer, tracheal cancer, urogenital cancer, and uterine cancer.

According to some embodiments, the present disclosure includes methods for treating, delaying, or inhibiting the growth of a tumor. In some embodiments, the present disclosure includes methods to promote tumor regression. In some embodiments, the present disclosure includes methods to reduce tumor cell load or to reduce tumor burden. In some embodiments, the present disclosure includes methods to prevent tumor recurrence.

The methods of the present disclosure, according to some embodiments, comprise administering to a subject in need thereof an oncolytic virus, a PD-1 pathway inhibitor (e.g., anti-PD-1 antibody or antigen-binding fragment thereof), or a CTLA4 inhibitor (e.g., anti-CTLA4 antibody or antigen-binding fragment thereof).

In some embodiments, the methods comprise administering to the subject one or more doses of an oncolytic virus before, after or concurrently with administering to the subject one or more doses of a PD-1 pathway inhibitor and/or one or more doses of a CTLA4 inhibitor. In some embodiments, one or more doses of the PD-1 pathway inhibitor can be administered in combination with one or more doses of the CTLA4 inhibitor.

As used herein, the term “in combination with” also includes sequential or concomitant administration of the oncolytic virus, the PD-1 pathway inhibitor (e.g., anti-PD-1 antibody or antigen-binding fragment thereof), and the CTLA4 inhibitor (e.g., anti-CTLA4 antibody or antigen-binding fragment thereof). For example, when administered “before” the CTLA4 inhibitor, one or more doses of the PD-1 pathway inhibitor (e.g., anti-PD-1 antibody or antigen-binding fragment thereof) may be administered more than about 12 weeks, about 11 weeks, about 10 weeks, about 9 weeks, about 8 weeks, about 7 weeks, about 6 weeks, about 5 weeks, about 4 weeks, about 3 weeks, about 2 weeks, about 150 hours, about 150 hours, about 100 hours, about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of one or more doses of the CTLA inhibitor.

When administered “after” the CTLA4 inhibitor (e.g., anti-CTLA4 antibody or antigen-binding fragment thereof), the PD-1 pathway inhibitor (e.g., anti-PD-1 antibody or antigen-binding fragment thereof) may be administered about 12 weeks, about 11 weeks, about 10 weeks, about 9 weeks, about 8 weeks, about 7 weeks, about 6 weeks, about 5 weeks, about 4 weeks, about 3 weeks, about 2 weeks, about 150 hours, about 150 hours, about 100 hours, about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes after the administration of the CTLA4 inhibitor.

Administration “concurrent” with the CTLA4 inhibitor (e.g., anti-CTLA4 antibody or antigen-binding fragment thereof) means that the PD-1 pathway inhibitor (e.g., anti-PD-1 antibody or antigen-binding fragment thereof) is administered to the subject in a separate dosage form within less than 10 minutes (before, after, or at the same time) of administration of the CTLA4 inhibitor or administered to the subject as a single combined dosage formulation comprising both the PD-1 pathway inhibitor and the CTLA4 inhibitor.

In some embodiments, the disclosed methods may further include administering an anti-tumor therapy. Anti-tumor therapies include, but are not limited to, conventional anti-tumor therapies such as chemotherapy, radiation, surgery, or as elsewhere described herein.

In some embodiments, the treatment produces a therapeutic effect selected from one or more of: delay in tumor growth, reduction in tumor cell number, tumor regression, increase in survival, partial response, and complete response. In some embodiments, the tumor growth in the patient is delayed by at least 10 days as compared to tumor growth in an untreated patient. In some embodiments, the tumor growth is inhibited by at least 20% (e.g., at least 30%, at least 40%, at least 50%, at least 60%, at least 70%, at least 80%, at least 90%, at least 100%, at least 200%, at least 300%) as compared to an untreated patient. In some embodiments, the tumor growth is inhibited by at least 20% (e.g., at least 30%, at least 40%, at least 50%, at least 60%, at least 70%, at least 80%, at least 90%, at least 100%, at least 200%, at least 300%) as compared to a patient administered the oncolytic virus, the PD-1 pathway inhibitor, or the CTLA4 inhibitor as monotherapy. In some embodiments, the tumor growth is inhibited by at least 20% (e.g., at least 30%, at least 40%, at least 50%, at least 60%, at least 70%, at least 80%, at least 90%, at least 100%, at least 200%, at least 300%) as compared to a patient administered two of the oncolytic virus, the PD-1 pathway inhibitor, and the CTLA4 inhibitor.

### Oncolytic Viruses

Oncolytic viruses are cancer therapies that employ engineered or naturally evolved viruses of cancer tropism to incite tumor cell death in the treated patient. In general, when a replicating oncolytic virus is administered, infected tumor cells have the potential to produce progeny virus, allowing destructive infection to spread to neighboring tumor cells. The potential for viral replication is determined by the cell's ability to sense and respond to the viral infection. Besides, oncolytic viruses bear pathogen-associated molecular patterns (PAMPs) that can act as adjuvants to stimulate myeloid cells (macrophages and dendritic cells) to enhance T cell stimulation.

In some embodiments, the oncolytic virus is a replication competent oncolytic rhabdovirus. Such oncolytic rhabdoviruses include, without limitation, wild type or genetically modified Arajas virus, Chandipura virus, Cocal virus, Isfahan virus, Maraba virus, Piry virus, Vesicular stomatitis Alagoas virus, Vesicular stomatitis virus (VSV), BeAn 157575 virus, Boteke virus, Calchaqui virus, Eel virus American, Gray Lodge virus, Jurona virus, Klamath virus, Kwatta virus, La Joya virus, Malpais Spring virus, Mount Elgon bat virus, Perinet virus, Tupaia virus, Farmington, Bahia Grande virus, Muir Springs virus, Reed Ranch virus, Hart Park virus, Flanders virus, Kamese virus, Mosqueiro virus, Mossuril virus, Barur virus, Fukuoka virus, Kern Canyon virus, Nkolbisson virus, Le Dantec virus, Keuraliba virus, Connecticut virus, New Minto virus, Sawgrass virus, Chaco virus, Sena Madureira virus, Timbo virus, Almpiwar virus, Aruac virus, Bangoran virus, Bimbo virus, Bivens Arm virus, Blue crab virus, Charleville virus, Coastal Plains virus, DakArK 7292 virus, Entamoeba virus, Garba virus, Gossas virus, Humpty Doo virus, Joinjakaka virus, Kannamangalam virus, Kolongo virus, Koolpinyah virus, Kotonkon virus, Landjia virus, Manitoba virus, Marco virus, Nasoule virus, Navarro virus, Ngaingan virus, Oak-Vale vims, Obodhiang vims, Oita vims, Ouango vims, Parry Creek vims, Rio Grande ci chlid vims, Sandjimba vims, Sigma vims, Sripur vims, Sweetwater Branch vims, Tibrogargan vims, Xiburema vims, Yata vims, Rhode Island, Adelaide River vims, Berrimah vims, Kimberley vims, or Bovine ephemeral fever vims.

Vesicular stomatitis virus (VSV), as indicated above, is a member of the Rhabdoviridae family. The VSV genome is a single molecule of negative-sense RNA that encodes 5 major polypeptides: a nucleocapsid (N) polypeptide, a phosphoprotein (P) polypeptide, a matrix (M) polypeptide, a glycoprotein (G) polypeptide, and a viral polymerase (L) polypeptide.

In some embodiments, the oncolytic virus is a wild type or recombinant VSV. In some embodiments, the recombinant VSV comprises one or more mutations, such as an M51R substitution (also herein referred to as VSV-M51R).

In some embodiments, the oncolytic virus may be engineered to express one or more cytokines, such as interferon-beta (IFNb). In some embodiments, IFNb (e.g., interferon beta-1a) can be a human or mouse IFNb or a variant thereof. In some embodiments, IFNb comprises an amino acid sequence having at least 90% (e.g., 90%, 95%, 96%, 97%, 98%, 99%) sequence identity to the amino acid sequence of SEQ ID NO: 23 or 24, or comprises the amino acid sequence of SEQ ID NO: 23 or 24. In some embodiments, a nucleic acid sequence encoding the IFNb is positioned between M and G viral genes. Such a position allows the virus to express an amount of IFNb polypeptide that is effective to activate anti-viral immune responses in non-cancerous tissue, and thus alleviate potential viral toxicity without impeding efficient viral replication in cancer cells.

In some embodiments, the recombinant VSV further expresses a sodium/iodide symporter (NIS) or a variant thereof. In some embodiments, the NIS comprises an amino acid sequence having at least 90% (e.g., 90%, 95%, 96%, 97%, 98%, 99%) sequence identity to the amino acid sequence of SEQ ID NO: 25 or comprises the amino acid sequence of SEQ ID NO: 25. In some embodiments, a nucleic acid sequence encoding the NIS is positioned between G and L viral genes which allows appropriate expression levels of NIS polypeptides.

In certain embodiments, the oncolytic virus is a recombinant VSV known in the art as Voyager V1 described in, e.g., U.S. Pat. No. 9,428,736, which is hereby incorporated by reference in its entirety.

### PD-1 Pathway Inhibitors

The methods disclosed herein include administering a therapeutically effective amount of a PD-1 pathway inhibitor. As used herein, a “PD-1 pathway inhibitor” refers to any molecule capable of inhibiting, blocking, abrogating or interfering with the activity or expression of PD-1. In some embodiments, the PD-1 pathway inhibitor can be an antibody, a small molecule compound, a nucleic acid, a polypeptide, or a functional fragment or variant thereof. Non-limiting examples of suitable PD-1 pathway inhibitors include anti-PD-1 antibodies and antigen-binding fragments thereof, anti-PD-L1 antibodies and antigen-binding fragments thereof, and anti-PD-L2 antibodies and antigen-binding fragments thereof.

Other non-limiting examples of suitable PD-1 pathway inhibitors include RNAi molecules such as anti-PD-1 RNAi molecules, anti-PD-L1 RNAi, and anti-PD-L2 RNAi, antisense molecules such as anti-PD-1 antisense RNA, anti-PD-L1 antisense RNA, and anti-PD-L2 antisense RNA, and dominant negative proteins such as a dominant negative PD-1 protein, a dominant negative PD-L1 protein, and a dominant negative PD-L2 protein. Some examples of the foregoing PD-1 pathway inhibitors are described in, e.g., U.S. Pat. No. 9,308,236, U.S. Ser. No. 10/011,656, and US 20170290808, the portions of which that identify PD-1 pathway inhibitors are hereby incorporated by reference.

The term “antibody,” as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g., IgM) or antigen-binding fragments thereof. Each heavy chain comprises a heavy chain variable region (“HCVR” or “VH”) and a heavy chain constant region (comprised of domains CH1, CH2, and CH3). Each light chain comprises a light chain variable region (“LCVR or “VL”) and a light chain constant region (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some embodiments, the FRs of the antibody (or antigen-binding fragment thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules.

As used herein, the terms “antigen-binding fragment” of an antibody, “antigen-binding portion” of an antibody, and the like, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In some embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}- C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The antibodies used in the methods disclosed herein may be human antibodies. As used herein, the term “human antibody” refers to antibodies having variable and constant regions derived from human germline immunoglobulin sequences. The human antibodies of the present disclosure may nonetheless include amino acid residues not encoded by human germline immunoglobulin sequences (e.g., mutations introduced by random or site-specific mutagenesis in vitro or by somatic mutation in vivo), for example, in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods disclosed herein may be recombinant human antibodies. As used herein, the term “recombinant human antibody” includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see, e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In some embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

### Anti-PD-1 Antibodies and Antigen-Binding Fragments Thereof

In some embodiments, PD-1 pathway inhibitors used in the methods disclosed herein are antibodies or antigen-binding fragments thereof that specifically bind PD-1 (e.g., anti-PD-1 antibodies). The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” PD-1, as used in the context of the present disclosure, includes antibodies that bind PD-1 or a portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human PD-1 may, however, have cross-reactivity to other antigens, such as PD-1 molecules from other (non-human) species.

According to certain exemplary embodiments, the anti-PD-1 antibody, or antigen-binding fragment thereof comprises a heavy chain variable region (HCVR), light chain variable region (LCVR), and/or complementarity determining regions (CDRs) comprising the amino acid sequences of any of the anti-PD-1 antibodies set forth in U.S. Pat. No. 9,987,500, which is hereby incorporated by reference in its entirety.

In certain exemplary embodiments, the anti-PD-1 antibody or antigen-binding fragment thereof that can be used in the context of the present disclosure comprises the heavy chain complementarity determining regions (HCDRs) of a heavy chain variable region (HCVR) comprising the amino acid sequence of SEQ ID NO: 1 and the light chain complementarity determining regions (LCDRs) of a light chain variable region (LCVR) comprising the amino acid sequence of SEQ ID NO: 2.

According to some embodiments, the anti-PD-1 antibody or antigen-binding fragment thereof comprises three HCDRs (HCDR1, HCDR2, and HCDR3) and three LCDRs (LCDR1, LCDR2, and LCDR3), wherein the HCDR1 comprises the amino acid sequence of SEQ ID NO: 3; the HCDR2 comprises the amino acid sequence of SEQ ID NO: 4; the HCDR3 comprises the amino acid sequence of SEQ ID NO: 5; the LCDR1 comprises the amino acid sequence of SEQ ID NO: 6; the LCDR2 comprises the amino acid sequence of SEQ ID NO: 7; and the LCDR3 comprises the amino acid sequence of SEQ ID NO: 8.

In yet other embodiments, the anti-PD-1 antibody or antigen-binding fragment thereof comprises an HCVR comprising SEQ ID NO: 1 and an LCVR comprising SEQ ID NO: 2. In some embodiments, the antibody comprises a heavy chain comprising the amino acid sequence of SEQ ID NO: 9. In some embodiments, the anti-PD-1 antibody comprises a light chain comprising the amino acid sequence of SEQ ID NO: 10.

An exemplary antibody comprising a heavy chain variable region comprising the amino acid sequence of SEQ ID NO: 1 and a light chain variable region comprising the amino acid sequence of SEQ ID NO: 2 is the fully human anti-PD-1 antibody known as cemiplimab (also known as REGN2810; LIBTAYO®).

According to certain exemplary embodiments, the methods of the present disclosure comprise the use of cemiplimab or a bioequivalent thereof. As used herein, the term “bioequivalent” with respect to a PD-1 pathway inhibitor refers to anti-PD-1 antibodies or PD-1-binding proteins or fragments thereof that are pharmaceutical equivalents or pharmaceutical alternatives whose rate and/or extent of absorption do not show a significant difference with that of a reference antibody (e.g., cemiplimab) when administered at the same molar dose under similar experimental conditions, either single dose or multiple doses. In the context of the present disclosure, the term “bioequivalent” includes antigen-binding proteins that bind to PD-1 and do not have clinically meaningful differences with cemiplimab with respect to safety, purity and/or potency.

According to some embodiments of the present disclosure, the anti-human PD-1, or antigen-binding fragment thereof, comprises a HCVR having at least 90% (e.g., 90%, 95%, 96%, 97%, 98%, 99%) sequence identity to SEQ ID NO: 1.

According to some embodiments of the present disclosure, the anti-human PD-1, or antigen-binding fragment thereof, comprises a LCVR having (e.g., 90%, 95%, 96%, 97%, 98%, 99%) sequence identity to SEQ ID NO: 2. Sequence identity may be measured by methods known in the art (e.g., GAP, BESTFIT, and BLAST).

According to some embodiments of the present disclosure, the anti-human PD-1 or antigen-binding fragment thereof comprises a HCVR comprising an amino acid sequence of SEQ ID NO: 1 having no more than 10 amino acid substitutions. According to some embodiments of the present disclosure, the anti-human PD-1 or antigen-binding fragment thereof comprises a LCVR comprising an amino acid sequence of SEQ ID NO: 2 having no more than 10 amino acid substitutions.

Also within the scope of this disclosure are variants of any of the HCVR, LCVR and/or CDR amino acid sequences disclosed herein having one or more conservative amino acid substitutions. For example, the present disclosure includes use of anti-PD-L1 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

Other anti-PD-1 antibodies or antigen-binding fragments thereof that can be used in the context of the methods of the present disclosure include, e.g., the antibodies referred to and known in the art as nivolumab, pembrolizumab, MED10608, pidilizumab, BI 754091, spartalizumab (also known as PDR001), camrelizumab (also known as SHR-1210), JNJ-63723283, MCLA-134, or any of the anti-PD-1 antibodies set forth in U.S. Pat. Nos. 6,808,710, 7,488,802, 8,008,449, 8,168,757, 8,354,509, 8,609,089, 8,686,119, 8,779,105, 8,900,587, and 9,987,500, and in patent publications WO 2006/121168, WO 2009/114335. The portions of all of the aforementioned publications that identify anti-PD-1 antibodies are hereby incorporated by reference.

The anti-PD-1 antibodies used in the context of the methods of the present disclosure may have pH-dependent binding characteristics. For example, an anti-PD-1 antibody for use in the methods of the present disclosure may exhibit reduced binding to PD-1 at acidic pH as compared to neutral pH. Alternatively, an anti-PD-1 antibody of the present disclosure may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to PD-1 at acidic pH as compared to neutral pH” is expressed in terms of a ratio of the K_{D} value of the antibody binding to PD-1 at acidic pH to the K_{D} value of the antibody binding to PD-1 at neutral pH (or vice versa). For example, an antibody or antigen-binding fragment thereof may be regarded as exhibiting “reduced binding to PD-1 at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained. As used herein, the expression “acidic pH” means a pH of 6.0 or less.

### Anti-PD-L1 Antibodies and Antigen-Binding Fragments Thereof

In some embodiments, PD-1 pathway inhibitors used in the methods disclosed herein are antibodies or antigen-binding fragments thereof that specifically bind PD-L1 (e.g., anti-PD-L1 antibodies). For example, an antibody that “specifically binds” PD-L1, as used in the context of the present disclosure, includes antibodies that bind PD-L1 or a portion thereof with a K_{D} of about 1×10⁻⁸ M or less (e.g., a smaller K_{D} denotes a tighter binding). A “high affinity” anti-PD-L1 antibody refers to those mAbs having a binding affinity to PD-L1, expressed as K_{D} of at least 10⁻⁸ M, such as 10⁻⁹ M, 10⁻¹⁰ M, 10⁻¹¹ M, or 10⁻¹² M, as measured by surface plasmon resonance, e.g., BIACORE™ or solution-affinity ELISA. An isolated antibody that specifically binds human PD-L1 may, however, have cross-reactivity to other antigens, such as PD-L1 molecules from other (non-human) species.

According to certain exemplary embodiments, the anti-PD-L1 antibody or antigen-binding fragment thereof comprises a heavy chain variable region (HCVR), light chain variable region (LCVR), and/or complementarity determining regions (CDRs) comprising the amino acid sequences of any of the anti-PD-L1 antibodies set forth in U.S. Pat. No. 9,938,345, which is hereby incorporated by reference in its entirety.

In certain exemplary embodiments, an anti-PD-L1 antibody or antigen-binding fragment thereof that can be used in the context of the present disclosure comprises the heavy chain complementarity determining regions (HCDRs) of a heavy chain variable region (HCVR) comprising SEQ ID NO: 11 and the light chain complementarity determining regions (LCDRs) of a light chain variable region (LCVR) comprising SEQ ID NO: 12. An exemplary anti-PD-L1 antibody comprising a HCVR of SEQ ID NO: 11 and a LCVR of SEQ ID NO: 12 is REGN3504.

According to some embodiments of the present disclosure, the anti-human PD-L1 antibody, or antigen-binding fragment thereof, comprises a HCVR having at least 90% (e.g., 90%, 95%, 96%, 97%, 98%, 99%) sequence identity to SEQ ID NO: 11. According to some embodiments of the present disclosure, the anti-human PD-L1 antibody, or antigen-binding fragment thereof, comprises a LCVR having at least 90% (e.g., 90%, 95%, 96%, 97%, 98%, 99%) sequence identity to SEQ ID NO: 12.

According to some embodiments of the present disclosure, the anti-human PD-L1 antibody, or antigen-binding fragment thereof, comprises a HCVR comprising an amino acid sequence of SEQ ID NO: 11 having no more than 10 amino acid substitutions. According to some embodiments of the present disclosure, the anti-human PD-L1 antibody, or antigen-binding fragment thereof, comprises a LCVR comprising an amino acid sequence of SEQ ID NO: 12 having no more than 10 amino acid substitutions.

Also within the scope of this disclosure are variants of any of the HCVR, LCVR and/or CDR amino acid sequences disclosed herein having one or more conservative amino acid substitutions. For example, the present disclosure includes use of anti-PD-L1 antibodies having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein.

Other anti-PD-L1 antibodies that can be used in the context of the methods of the present disclosure include, e.g., the antibodies referred to and known in the art as MDX-1105, atezolizumab (TECENTRIQ™), durvalumab (IMFINZI™), avelumab (BAVENCIO™) LY3300054, FAZ053, STI-1014, CX-072, KN035 (Zhang et al., Cell Discovery, 3, 170004 (March 2017)), CK-301 (Gorelik et al., American Association for Cancer Research Annual Meeting (AACR), 2016 Apr. 4 Abstract 4606), or any of the other anti-PD-L1 antibodies set forth in U.S. Pat. Nos. 7,943,743, 8,217,149, 9,402,899, 9,624,298, and 9,938,345, and in patent publications WO 2007/005874, WO 2010/077634, WO 2013/181452, WO 2013/181634, WO 2016/149201, WO 2017/034916, or EP3177649. The portions of all of the aforementioned publications that identify anti-PD-L1 antibodies are hereby incorporated by reference.

### Anti-PD-L2 Antibodies and Antigen-Binding Fragments Thereof

In some embodiments, PD-1 pathway inhibitors used in the methods disclosed herein are antibodies or antigen-binding fragments thereof that specifically bind PD-L2 (e.g., anti-PD-L2 antibodies). For example, an antibody that “specifically binds” PD-L2, as used in the context of the present disclosure, includes antibodies that bind PD-L2 or a portion thereof with a KD of about 1×10⁻⁸ M or less (e.g., a smaller KD denotes a tighter binding). A “high affinity” anti-PD-L2 antibody refers to those mAbs having a binding affinity to PD-L2, expressed as KD of at least 10-8 M, such as 10⁻⁹ M, 10⁻¹⁰ M, 10⁻¹¹ M, or 10⁻¹² M, as measured by surface plasmon resonance, e.g., BIACORE™ or solution-affinity ELISA. An isolated antibody that specifically binds human PD-L2 may, however, have cross-reactivity to other antigens, such as PD-L2 molecules from other (non-human) species.

Anti-PD-L2 antibodies that can be used in the context of the methods of the present disclosure include, e.g., the anti-PD-L2 antibodies set forth in U.S. Pat. No. 8,552,154 and 10,647,771. The portions of all of the aforementioned publications that identify anti-PD-L2 antibodies are hereby incorporated by reference.

### CTLA4 Inhibitors

The methods disclosed herein include administering a therapeutically effective amount of a CTLA4 inhibitor. As used herein, a “CTLA4 inhibitor” refers to any molecule capable of inhibiting, blocking, abrogating or interfering with the activity or expression of CTLA4. In some embodiments, the CTLA4 inhibitor can be an antibody, a small molecule compound, a nucleic acid, a polypeptide, or a functional fragment or variant thereof. Non-limiting examples of suitable CTLA4 inhibitors include anti-CTLA4 antibodies and antigen-binding fragments thereof. Other non-limiting examples of suitable CTLA4 inhibitors include RNAi molecules such as anti-CTLA4 RNAi molecules and dominant negative proteins such as a dominant negative CTLA4 protein.

### Anti-CTLA4 Antibodies and Antigen-Binding Fragments Thereof

In some embodiments, CTLA4 inhibitors used in the methods disclosed herein are antibodies or antigen-binding fragments thereof that specifically bind CTLA4 (e.g., anti-CTLA4 antibodies). The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” CTLA4, as used in the context of the present disclosure, includes antibodies that bind CTLA4 antibody or a portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human CTLA4 may, however, have cross-reactivity to other antigens, such as CTLA4 molecules from other (non-human) species.

In certain exemplary embodiments, the anti-CTLA4 antibody or antigen-binding fragment thereof that can be used in the context of the present disclosure comprises the heavy chain complementarity determining regions (HCDRs) of a heavy chain variable region (HCVR) comprising the amino acid sequence of SEQ ID NO: 13 and the light chain complementarity determining regions (LCDRs) of a light chain variable region (LCVR) comprising the amino acid sequence of SEQ ID NO: 14.

According to some embodiments, the anti-CTLA4 antibody or antigen-binding fragment thereof comprises three HCDRs (HCDR1, HCDR2, and HCDR3) and three LCDRs (LCDR1, LCDR2, and LCDR3), wherein the HCDR1 comprises the amino acid sequence of SEQ ID NO: 15; the HCDR2 comprises the amino acid sequence of SEQ ID NO: 16; the HCDR3 comprises the amino acid sequence of SEQ ID NO: 17; the LCDR1 comprises the amino acid sequence of SEQ ID NO: 18; the LCDR2 comprises the amino acid sequence of SEQ ID NO: 19; and the LCDR3 comprises the amino acid sequence of SEQ ID NO: 20.

In yet other embodiments, the anti-CTLA4 antibody or antigen-binding fragment thereof comprises an HCVR comprising the amino acid sequence of SEQ ID NO: 13 and an LCVR comprising the amino acid sequence of SEQ ID NO: 14. In some embodiments, the antibody comprises a heavy chain comprising the amino acid sequence of SEQ ID NO: 21. In some embodiments, the anti-CTLA4 antibody comprises a light chain comprising the amino acid sequence of SEQ ID NO: 22.

An exemplary antibody comprising a heavy chain variable region comprising the amino acid sequence of SEQ ID NO: 13 and a light chain variable region comprising the amino acid sequence of SEQ ID NO: 14 is the fully human anti-CTLA4 antibody known as REGN4659.

According to certain exemplary embodiments, the methods of the present disclosure comprise the use of REGN4659 or a bioequivalent thereof. As used herein, the term “bioequivalent” with respect to a CTLA4 inhibitor refers to anti-CTLA4 antibodies or CTLA4-binding proteins or fragments thereof that are pharmaceutical equivalents or pharmaceutical alternatives whose rate and/or extent of absorption do not show a significant difference with that of a reference antibody (e.g., REGN4659) when administered at the same molar dose under similar experimental conditions, either single dose or multiple doses. In the context of the present disclosure, the term “bioequivalent” includes antigen-binding proteins that bind to CTLA4 and do not have clinically meaningful differences with REGN4659 with respect to safety, purity and/or potency.

According to some embodiments of the present disclosure, the anti-human CTLA4, or antigen-binding fragment thereof, comprises a HCVR having at least 90% (e.g., 90%, 95%, 96%, 97%, 98%, 99%) sequence identity to the amino acid sequence of SEQ ID NO: 13.

According to some embodiments of the present disclosure, the anti-human CTLA4, or antigen-binding fragment thereof, comprises a LCVR having (e.g., 90%, 95%, 96%, 97%, 98%, 99%) sequence identity to the amino acid sequence of SEQ ID NO: 14.

According to some embodiments of the present disclosure, the anti-human CTLA4, or antigen-binding fragment thereof, comprises a HCVR comprising an amino acid sequence of SEQ ID NO: 13 having no more than 10 amino acid substitutions. According to some embodiments of the present disclosure, the anti-human CTLA4, or antigen-binding fragment thereof, comprises a LCVR comprising an amino acid sequence of SEQ ID NO: 14 having no more than 10 amino acid substitutions.

Also within the scope of this disclosure are variants of any of the HCVR, LCVR and/or CDR amino acid sequences disclosed herein having one or more conservative amino acid substitutions. For example, the present disclosure includes use of anti-PD-L1 antibodies having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein.

Other anti-CTLA4 antibodies or antigen-binding fragments thereof that can be used in the context of the methods of the present disclosure include, e.g., the antibodies referred to and known in the art as ipilimumab, tremelimumab, or any of the anti-CTLA4 antibodies set forth in U.S. Pat. Nos. 7,527,969, 8,779,098, 7,666,424, 7,737,258, 7,740,845, 8,148,154, 8,414,892, 8,501,471, and 9,062,110; and in patent publications US2013/0078234, US2010/0143245, WO2017062615A2, WO 2004/001381, and WO 2012/147713. The portions of all of the aforementioned publications that identify anti-CTLA4 antibodies are hereby incorporated by reference.

### Pharmaceutical Compositions and Administration

The present disclosure includes methods which comprise administering an oncolytic virus, a PD-1 pathway inhibitor, and/or a CTLA4 inhibitor to a subject wherein the antibodies are contained within a separate or combined (single) pharmaceutical composition. The pharmaceutical compositions of this disclosure may be formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN), DNA conjugates, anhydrous absorption pastes, oil-in-water, and water-in-oil emulsions, emulsions carbowax (polyethylene glycols of various molecular weights), semi-solid gels, and semi-solid mixtures containing carbowax. See also Powell et al. PDA (1998) J Pharm Sci Technol 52:238-311.

Various delivery systems are known and can be used to administer the pharmaceutical composition of the present disclosure, e.g., encapsulation in liposomes, microparticles, microcapsules, recombinant cells capable of expressing the mutant viruses, receptor-mediated endocytosis (see, e.g., Wu et al., 1987, J. Biol. Chem. 262: 4429-4432). Methods of administration include, but are not limited to, intradermal, intramuscular, intratumoral, intraperitoneal, intravenous, subcutaneous, intranasal, epidural, and oral routes. The composition may be administered by any convenient route, for example, by infusion or bolus injection, by absorption through epithelial or mucocutaneous linings (e.g., oral mucosa, rectal and intestinal mucosa, etc.) and may be administered together with other biologically active agents.

A pharmaceutical composition comprising an oncolytic virus, a PD-1 pathway inhibitor, or a CTLA4 inhibitor can be delivered intratumorally, subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered, and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used. In another embodiment, polymeric materials can be used; see, e.g., Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous, intratumor and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

The present disclosure also provides kits comprising an oncolytic virus, a PD-1 pathway inhibitor, and a CTLA4 inhibitor, in combination with written instructions for use of a therapeutically effective amount of a combination of the oncolytic virus, the PD-1 pathway inhibitor, and the CTLA4 inhibitor for treating or inhibiting the growth of a tumor of a patient. Administration Regimens

The methods of the present disclosure may include administering to a subject an oncolytic virus, a PD-1 pathway inhibitor (e.g., an anti-PD-1, anti-PD-L1, or anti-PD-L2 antibody, or antigen-binding fragment thereof), or a CTLA4 inhibitor (e.g., anti-CTLA4 antibody or antigen-binding fragment thereof) at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. The methods of the present disclosure may also include administering a single dose each of an oncolytic virus, a PD-1 pathway inhibitor, or a CTLA4 inhibitor.

In some embodiments, at least one of the oncolytic virus, the PD-1 pathway inhibitor, or the CTLA4 inhibitor is administered to the patient once a day, once every two days, once every three days, once every four days, once every five days, once every week, once every two weeks, or once every three weeks.

In some embodiments, the oncolytic virus, the PD-1 pathway inhibitor, and the CTLA4 inhibitor are administered concurrently to the patient.

In some embodiments, the methods may include sequentially administering to the subject two or more of the oncolytic virus, the PD-1 pathway inhibitor, and the CTLA4 inhibitor. In some embodiments, the oncolytic virus is administered to the patient before or after the PD-1 pathway inhibitor and the CTLA4 inhibitor. In some embodiments, the PD-1 pathway inhibitor is administered to the patient before or after the oncolytic virus and the CTLA4 inhibitor. In some embodiments, the CTLA4 inhibitor is administered to the patient before or after the oncolytic virus and the PD-1 pathway inhibitor.

As used herein, “sequentially administering” means that each dose of the oncolytic virus, the PD-1 pathway inhibitor, or the CTLA4 inhibitor is administered to the subject at a different point in time, e.g., on different days separated by a predetermined interval (e.g., hours, days, weeks or months). The present disclosure includes methods which comprise sequentially administering to the patient a single initial dose of the oncolytic virus, the PD-1 pathway inhibitor, or the CTLA4 inhibitor, followed by one or more secondary doses of the oncolytic virus, the PD-1 pathway inhibitor, or the CTLA4 inhibitor, and optionally followed by one or more tertiary doses of the oncolytic virus, the PD-1 pathway inhibitor, or the CTLA4 inhibitor. In some embodiments, the methods further comprise sequentially administering to the patient a single initial dose of the oncolytic virus, the PD-1 pathway inhibitor, or the CTLA4 inhibitor, followed by one or more secondary doses of the oncolytic virus, the PD-1 pathway inhibitor, or the CTLA4 inhibitor, and optionally followed by one or more tertiary doses the oncolytic virus, the PD-1 pathway inhibitor, or the CTLA4 inhibitor.

The terms “initial dose,” “secondary doses,” and “tertiary doses,” refer to the temporal sequence of administration. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the oncolytic virus, the PD-1 pathway inhibitor, or the CTLA4 inhibitor. In some embodiments, however, the amount contained in the initial, secondary, and/or tertiary doses varies from one another (e.g., adjusted up or down as appropriate) during the course of treatment. In some embodiments, one or more (e.g., 1, 2, 3, 4, or 5) doses are administered at the beginning of the treatment regimen as “loading doses” followed by subsequent doses that are administered on a less frequent basis (e.g., “maintenance doses”).

In one exemplary embodiment of the present disclosure, each secondary and/or tertiary dose is administered ½ to 14 (e.g., ½, 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, a dose of the oncolytic virus, the PD-1 pathway inhibitor, or the CTLA4 inhibitor, which is administered to a patient prior to the administration of the very next dose in the sequence with no intervening doses.

In some embodiments, the methods may include administering to a patient any number of secondary and/or tertiary doses of the oncolytic virus, the PD-1 pathway inhibitor (e.g., anti-PD-1 antibody or antigen-binding fragment thereof), or the CTLA4 inhibitor (e.g., anti-CTLA4 antibody or antigen-binding fragment thereof). For example, in some embodiments, only a single secondary dose is administered to the patient. In other embodiments, two or more (e.g., 2, 3, 4, 5, 6, 7, 8, or more) secondary doses are administered to the patient. Likewise, in some embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In some embodiments, one or more doses of the oncolytic virus, the PD-1 pathway inhibitor, or the CTLA4 inhibitor are administered at the beginning of a treatment regimen as “induction doses” on a more frequent basis (twice a week, once a week or once in 2 weeks) followed by subsequent doses (“consolidation doses” or “maintenance doses”) that are administered on a less frequent basis (e.g., once in 4-12 weeks).

### Dosage

The amount of the oncolytic virus, the PD-1 pathway inhibitor (e.g., an anti-PD-1 antibody or antigen-binding fragment thereof), or the CTLA4 inhibitor (e.g., an anti-CTLA4 antibody or antigen-binding fragment thereof) administered to a subject according to the methods disclosed herein is, generally, a therapeutically effective amount. As used herein, the term “therapeutically effective amount” means an amount of an oncolytic virus, a PD-1 pathway inhibitor, and/or a CTLA4 inhibitor that results in one or more of: (a) a reduction in the severity or duration of a symptom or an indication of cancer, e.g., a tumor lesion; (b) inhibition of tumor growth, or an increase in tumor necrosis, tumor shrinkage and/or tumor disappearance; (c) delay in tumor growth and development; (d) inhibition of tumor metastasis; (e) prevention of recurrence of tumor growth; (f) increase in survival of a subject with a cancer; and/or (g) a reduction in the use or need for conventional anti-cancer therapy (e.g., elimination of need for surgery or reduced or eliminated use of chemotherapeutic or cytotoxic agents) as compared to an untreated subject, a subject treated with monotherapy, or a subject treated with any two of the three therapeutic agents disclosed herein (PD-1 pathway inhibitor, CTLA4 inhibitor and the oncolytic virus).

In some embodiments, the oncolytic virus of the combination may be administered as one or more unit doses of 10, 100, 10³, 10⁴, 10⁵, 10⁶, 10⁷, 10⁸, 10⁹, 10¹⁰, 10¹¹, 10¹², 10¹³, 10¹⁴, or more viral particles (vp) or plaque-forming units (pfu). In some embodiments, the oncolytic virus is an oncolytic rhabdovirus (e.g., wild type or genetically modified VSV) and is administered to a human with cancer as one or more dosages of 10⁶-10¹⁴ pfu, 10⁶-10¹² pfu, 10⁸-10¹⁴ pfu, 10⁸-10¹² or 10¹⁰-10¹² pfu or any range therebetween.

In some embodiments, the oncolytic virus of the combination may be administered as one or more unit doses of 10, 100, 10³, 10⁴, 10⁵, 10⁶, 10⁷, 10⁸, 10⁹, 10¹⁰, 10¹¹, 10¹², 10¹³, 10¹⁴, or more 50% Tissue Culture Infectious Dose (TCID₅₀). In some embodiments, the oncolytic virus is an oncolytic rhabdovirus (e.g., wild type or genetically modified VSV) and is administered to a human with cancer as one or more dosages of 10⁴-10¹⁴ TCID₅₀, 10⁴-10¹⁴ TCID₅₀, 10⁴-10¹² TCID₅₀, 10⁸-10¹⁴ TCID₅₀, 10⁸-10¹² or 10¹⁰-10¹² TCID₅₀ or any range therebetween.

In some embodiments, a therapeutically effective amount of the PD-1 pathway inhibitor (e.g., an anti-PD-1 antibody or antigen-binding fragment thereof, such as cemiplimab or a bioequivalent thereof) can be from about 0.05 mg to about 1500 mg, from about 1 mg to about 800 mg, from about 5 mg to about 600 mg, from about 10 mg to about 550 mg, from about 50 mg to about 400 mg, from about 75 mg to about 350 mg, or from about 100 mg to about 300 mg of the antibody. For example, in various embodiments, the amount of the PD-1 pathway inhibitor is about 0.05 mg, about 0.1 mg, about 1.0 mg, about 1.5 mg, about 2.0 mg, about 5 mg, about 10 mg, about 15 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg, about 1090 mg, about 1200 mg, about 1210 mg, about 1220 mg, about 1230 mg, about 1240 mg, about 1250 mg, about 1260 mg, about 1270 mg, about 1280 mg, about 1290 mg, about 1300 mg, about 1310 mg, about 1320 mg, about 1330 mg, about 1340 mg, about 1350 mg, about 1360 mg, about 1370 mg, about 1380 mg, about 1390 mg, about 1400 mg, about 1410 mg, about 1420 mg, about 1430 mg, about 1440 mg, about 1450 mg, about 1460 mg, about 1470 mg, about 1480 mg, about 1490 mg, or about 1500 mg.

The amount of a PD-1 pathway inhibitor (e.g., an anti-PD-1 antibody or antigen-binding fragment thereof) contained within an individual dose may be expressed in terms of milligrams of antibody per kilogram of subject body weight (i.e., mg/kg). In some embodiments, the PD-1 pathway inhibitor used in the methods disclosed herein may be administered to a subject at a dose of about 0.0001 to about 100 mg/kg of subject body weight. In some embodiments, an anti-PD-1 antibody may be administered at a dose of about 0.1 mg/kg to about 20 mg/kg of a patient's body weight. In some embodiments, the methods of the present disclosure comprise administration of a PD-1 pathway inhibitor (e.g., an anti-PD-1 antibody or antigen-binding fragment thereof) at a dose of about 1 mg/kg to 3 mg/kg, 1 mg/kg to 5 mg/kg, 1 mg/kg to 10 mg/kg, 1 mg/kg, 3 mg/kg, 5 mg/kg, or 10 mg/kg of a patient's body weight.

In some embodiments, each dose comprises 0.1-10 mg/kg (e.g., 0.3 mg/kg, 1 mg/kg, 3 mg/kg, or 10 mg/kg) of the subject's body weight. In certain other embodiments, each dose comprises 5-1500 mg of the PD-1 pathway inhibitor (such as an anti-PD-1 antibody or antigen-binding fragment thereof), e.g., 5 mg, 10 mg, 15 mg, 20 mg, 25 mg, 30 mg, 40 mg, 45 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1550 mg, 1300 mg, 1350 mg, 1400 mg, 1450 mg, or 1500 mg of the PD-1 pathway inhibitor.

In some embodiments, a therapeutically effective amount of the CTLA4 inhibitor (e.g., an anti-CTLA4 antibody or antigen-binding fragment thereof, or a bioequivalent thereof) can be from about 0.05 mg to about 1000 mg, from about 1 mg to about 800 mg, from about 5 mg to about 600 mg, from about 10 mg to about 550 mg, from about 50 mg to about 400 mg, from about 75 mg to about 350 mg, or from about 100 mg to about 300 mg of the antibody. For example, in various embodiments, the amount of the CTLA4 inhibitor is about 0.05 mg, about 0.1 mg, about 1.0 mg, about 1.5 mg, about 2.0 mg, about 5 mg, about 10 mg, about 15 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, or about 1000 mg.

The amount of a CTLA4 inhibitor (e.g., an anti-CTLA4 antibody or antigen-binding fragment thereof) contained within an individual dose may be expressed in terms of milligrams of antibody per kilogram of subject body weight (i.e., mg/kg). In some embodiments, an anti-CTLA4 antibody may be administered at a dose of about 0.1 mg/kg to about 20 mg/kg of a patient's body weight. In some embodiments, the methods of the present disclosure comprise administration of a CTLA4 inhibitor (e.g., an anti-CTLA4 antibody or antigen-binding fragment thereof) at a dose of about 1 mg/kg to 3 mg/kg, 1 mg/kg to 5 mg/kg, 1 mg/kg to 10 mg/kg, 1 mg/kg, 3 mg/kg, 5 mg/kg, 10 mg/kg or 15 mg/kg of a patient's body weight.

In some embodiments, each dose comprises 0.1-10 mg/kg (e.g., 0.3 mg/kg, 1 mg/kg, 3 mg/kg, or 10 mg/kg) of the subject's body weight. In certain other embodiments, each dose comprises 5-1000 mg of the CTLA4 inhibitor (such as an anti-CTLA4 antibody or antigen-binding fragment thereof), e.g., 5 mg, 10 mg, 15 mg, 20 mg, 25 mg, 30 mg, 40 mg, 45 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg of the CTLA4 inhibitor.

In some embodiments, the methods of the present disclosure further include administering to a subject an additional therapeutic agent or therapy. The additional therapeutic agent or therapy may be administered for increasing anti-tumor efficacy, for reducing toxic effects of one or more therapies and/or for reducing the dosage of one or more therapies. In various embodiments, the additional therapeutic agent or therapy may include one or more of: radiation, surgery, a cancer vaccine, imiquimod, an anti-viral agent (e.g., cidofovir), photodynamic therapy, a lymphocyte activation gene 3 (LAG3) inhibitor (e.g., an anti-LAG3 antibody, a glucocorticoid-induced tumor necrosis factor receptor (GITR) agonist (e.g., an anti-GITR antibody), a T-cell immunoglobulin and mucin containing −3 (TIM3) inhibitor, a B- and T-lymphocyte attenuator (BTLA) inhibitor, a T-cell immunoreceptor with Ig and ITIM domains (TIGIT) inhibitor, a CD38 inhibitor, a CD47 inhibitor, an indoleamine-2,3-dioxygenase (IDO) inhibitor, a CD28 activator, a vascular endothelial growth factor (VEGF) antagonist (e.g., a “VEGF-Trap” such as aflibercept, or an anti-VEGF antibody or antigen-binding fragment thereof (e.g., bevacizumab, or ranibizumab) or a small molecule kinase inhibitor of VEGF receptor (e.g., sunitinib, sorafenib, or pazopanib)), an angiopoietin-2 (Ang2) inhibitor, a transforming growth factor beta (TGFβ) inhibitor, an epidermal growth factor receptor (EGFR) inhibitor, an antibody to a tumor-specific antigen (e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9), a vaccine (e.g., Bacillus Calmette-Guerin), granulocyte-macrophage colony-stimulating factor (GM-CSF), a second oncolytic virus, a cytotoxin, a chemotherapeutic agent (e.g., pemetrexed, dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, topotecan, irinotecan, vinorelbine, and vincristine), an IL-6R inhibitor, an IL-4R inhibitor, an IL-10 inhibitor, a cytokine such as IL-2, IL-7, IL-12, IL-21, and IL-15, an antibody drug conjugate, an anti-inflammatory drug such as a corticosteroid, a non-steroidal anti-inflammatory drug (NSAID), cryotherapy, anti-HPV therapy, laser therapy, electrosurgical excision of cells with HPV, and combinations thereof.

In some embodiments, the methods further comprise administering an additional therapeutic agent, such as an anti-cancer drug. As used herein, “anti-cancer drug” means any agent useful to treat cancer including, but not limited to, cytotoxins and agents such as antimetabolites, alkylating agents, anthracyclines, antibiotics, antimitotic agents, procarbazine, hydroxyurea, asparaginase, corticosteroids, mitotane (0, P′-(DDD)), biologics (e.g., antibodies and interferons) and radioactive agents. As used herein, “a cytotoxin or cytotoxic agent” also refers to a chemotherapeutic agent and means any agent that is detrimental to cells. Examples include TAXOL (paclitaxel), temozolomide, cytochalasin B, gramicidin D, ethidium bromide, emetine, cisplatin, mitomycin, etoposide, teniposide, vincristine, vinblastine, colchicine, doxorubicin, daunorubicin, dihydroxy anthracene dione, mitoxantrone, mithramycin, actinomycin D, 1-dihydrotestosterone, glucocorticoids, procaine, tetracaine, lidocaine, propranolol, and puromycin and analogs or homologs thereof.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

As used herein, the term “agent” denotes a chemical compound, a mixture of chemical compounds, a biological macromolecule (such as a nucleic acid, an antibody, a protein or portion thereof, e.g., a peptide), or an extract made from biological materials, such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject. In the context of the disclosure, the term “therapeutic agent” refers to any of the PD-1 pathway inhibitors, CTLA4 inhibitors or oncolytic viruses disclosed herein.

As used herein, the terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder or pathological condition.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

As used herein, the term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present disclosure within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of one or more components of the present disclosure and are physiologically acceptable to the subject. Supplementary active compounds may also be incorporated into the compositions.

Doses are often expressed in relation to bodyweight. Thus, a dose which is expressed as [g, mg, or other unit]/kg (or g, mg, etc.) usually refers to [g, mg, or other unit] “per kg (or g, mg, etc.) bodyweight,” even if the term “bodyweight” is not explicitly mentioned. The treatments may include various “unit doses.” A unit dose is defined as containing a predetermined quantity of the therapeutic composition. A unit dose need not be administered as a single injection but may comprise continuous infusion over a set period of time. For oncolytic viruses, a unit dose may be described in terms of plaque-forming units (pfu) or viral particles for viral constructs. Unit doses range from 10³, 10⁴, 10⁵, 10⁶, 10⁷, 10⁸, 10⁹, 10¹⁰, 10¹¹, 10¹², 10¹³ pfu or vp and higher. Alternatively, depending on the kind of virus and the titer attainable, one will deliver 1 to 100, 10 to 50, 100-1000, or up to about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, or 1×10¹⁵ or higher infectious viral particles (vp) to the patient or to the patient's cells. Alternatively, unit doses for oncolytic viruses are represented by TCID₅₀. “TCID₅₀” refers to “tissue culture infective dose” and is defined as the dilution of a virus required to infect 50% of a given batch of inoculated cell cultures. Various methods known to one skilled in the art may be used to calculate TCID₅₀, including the Spearman-Karber method which is utilized throughout this specification. For a description of the Spearman-Karber method, see B. W. Mahy & H. 0. Kangro, Virology Methods Manual 25-46 (1996). In some embodiments, Unit doses range from 10³, 10⁴, 10⁵, 10⁶, 10⁷, 10⁸, 10⁹, 10¹⁰, 10¹¹, 10¹², 10¹³ TCID₅₀ and higher or any ranges therebetween.

As used herein, the term “disease” is intended to be generally synonymous and is used interchangeably with the terms “disorder” and “condition” (as in medical condition), in that all reflect an abnormal condition (e.g., cancer) of the human or animal body or of one of its parts that impairs normal functioning, is typically manifested by distinguishing signs and symptoms, and causes the human or animal to have a reduced duration or quality of life.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

As used herein, the singular forms “a,” “an,” and “the” include plural references unless the context clearly dictates otherwise.

As used herein, the terms “including,” “comprising,” “containing,” or “having,” and variations thereof are meant to encompass the items listed thereafter and equivalents thereof as well as additional subject matter unless otherwise noted.

As used herein, the phrases “in one embodiment,” “in various embodiments,” “in some embodiments,” and the like are used repeatedly. Such phrases do not necessarily refer to the same embodiment, but they may unless the context dictates otherwise.

As used herein, the terms “and/or” or “/” means any one of the items, any combination of the items, or all of the items with which this term is associated.

As used herein, the word “substantially” does not exclude “completely,” e.g., a composition which is “substantially free” from Y may be completely free from Y. Where necessary, the word “substantially” may be omitted from the definition of the present disclosure.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

The use of any and all examples, or exemplary language (e.g., “such as”) provided herein, is intended merely to better illuminate the present disclosure and does not pose a limitation on the scope of the present disclosure unless otherwise claimed. No language in the specification should be construed as indicating any non-claimed element as essential to the practice of the present disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present disclosure is not entitled to antedate such publication by virtue of prior disclosure. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### Examples

The following examples are put forth so as to provide those of ordinary skill in the art with a complete disclosure and description of how to make and use the methods and compositions of the present disclosure and are not intended to limit the scope of what the inventors regard as their invention. Likewise, the disclosure is not limited to any particular preferred embodiments described herein. Indeed, modifications and variations of the embodiments may be apparent to those skilled in the art upon reading this specification and can be made without departing from its spirit and scope. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Anti-Tumor Efficacy of the Combination Treatment with Anti-PD-1, Anti-CTLA4, and Intra-Tumor Delivery of Oncolytic Virus VSV-M51R-Fluc in Mice Bearing 150 Mm³ Average MC38 Tumors

This example describes the anti-tumor efficacy of a triple combination using the oncolytic virus Vesicular Stomatitis Virus (VSV) with anti-PD-1 and anti-CTLA4 in wild type mice implanted with MC38 tumors. The VSV used in this example (as well as in subsequent Examples 2-3) is a genetically attenuated virus named VSV-M51R-Fluc as it encodes a mutation in the M protein (M51R) (M protein inhibits host cell protein production, but the M51R mutation preserves host cell protein production), and encodes for the firefly luciferase, inserted between the G and L viral genes. The anti-PD-1 antibody used in this example (as well as in subsequent Examples 2-4) is the anti-mouse PD-1 rat IgG2a antibody (clone 29F1.A12 from Bioxcell), and the anti-CTLA4 antibody used in this example (as well as in subsequent Examples 2-4) was anti-mouse CTLA4-mlgG2a antibody (clone 9D9).

C57BL/6 strain background mice from Jackson Laboratories were implanted subcutaneously with MC38 cells (3×10⁵ cells suspended in 100 μl of DMEM/mouse) at day 0. Tumors were measured using a caliper, and tumor volumes were calculated with the formula (L²×W)/2, where L is the smallest size. Mice were randomized evenly into 7 treatment groups when the average tumor size reached 150 mm³ which was at day 15. Mice were injected intratumorally with 50 μl of VSV-M51R-Fluc virus at 5×10⁵ TCID₅₀ dose resuspended in 50 μl PBS or PBS as control, and/or with an intraperitoneal injection of 250 μg of either isotype control antibody (mlgG2a and/or rat IgG2a) and/or 250 μg of the anti-CTLA4 antibody, and/or the anti-PD-1 antibody on days 15, 19, 22, and 26. Experimental dosing and treatment protocol for the various groups is shown in Table 1.

TABLE 1
Experimental dosing and
treatment protocol for groups of mice

| | | Virus | | | | | |
|---|---|---|---|---|---|---|---|
| | Virus | or | | | | Dosing | |
| | or | PBS | | Ab 1 | Ab 2 | interval | |
| | PBS | dosed | | (route | (route | Anti- | n |
| Group | Route | at D15 | TCID₅₀ | IP) | IP) | bodies | # |
| 1 | Intra- | PBS | — | Isotype | Isotype | D15, 19, | 5 |
| | tumor | | | rat | mouse | 22 and | |
| | | | | IgG2a | IgG2a | 26 | |
| | | | | 250 μg | 250 μg | | |
| 2 | Intra- | PBS | — | a-PD-1 | Isotype | D15, 19, | 5 |
| | tumor | | | 250 μg | mouse | 22 and | |
| | | | | | IgG2a | 26 | |
| | | | | | 250 μg | | |
| 3 | Intra- | PBS | — | a-PD-1 | a- | D15, 19, | 6 |
| | tumor | | | 250 μg | CTLA4 | 22 and | |
| | | | | | 250 μg | 26 | |
| 4 | Intra- | VSV- | 5 × 10⁵ | Isotype | Isotype | D15, 19, | 5 |
| | tumor | M51R- | TCID₅₀ | rat | mouse | 22 and | |
| | | Fluc | | IgG2a | IgG2a | 26 | |
| | | | | 250 μg | 250 μg | | |
| 5 | Intra- | VSV- | 5 × 10⁵ | a-PD-1 | Isotype | D15, 19, | 6 |
| | tumor | M51R- | TCID₅₀ | 250 μg | mouse | 22 and | |
| | | Fluc | | | IgG2a | 26 | |
| | | | | | 250 μg | | |
| 6 | Intra- | VSV- | 5 × 10⁵ | Isotype | a- | D15, 19, | 6 |
| | tumor | M51R- | TCID₅₀ | rat | CTLA4 | 22 and | |
| | | Flue | | IgG2a | 250 μg | 26 | |
| | | | | 250 μg | | | |
| 7 | Intra- | VSV- | 5 × 10⁵ | a-PD-1 | a- | D15, 19, | 6 |
| | tumor | M51R- | TCID₅₀ | 250 μg | CTLA4 | 22 and | |
| | | Flue | | | 250 μg | 26 | |

Tumor volumes were monitored by caliper measurements twice per week until the end of the study at day 60.

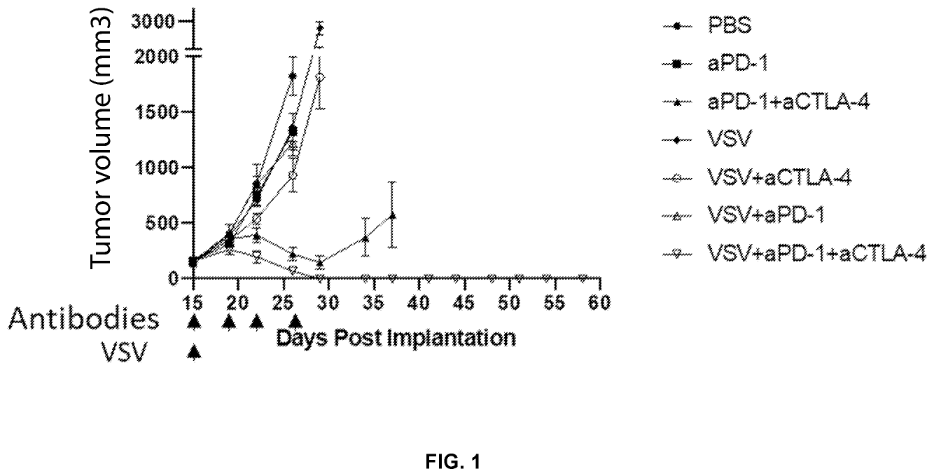

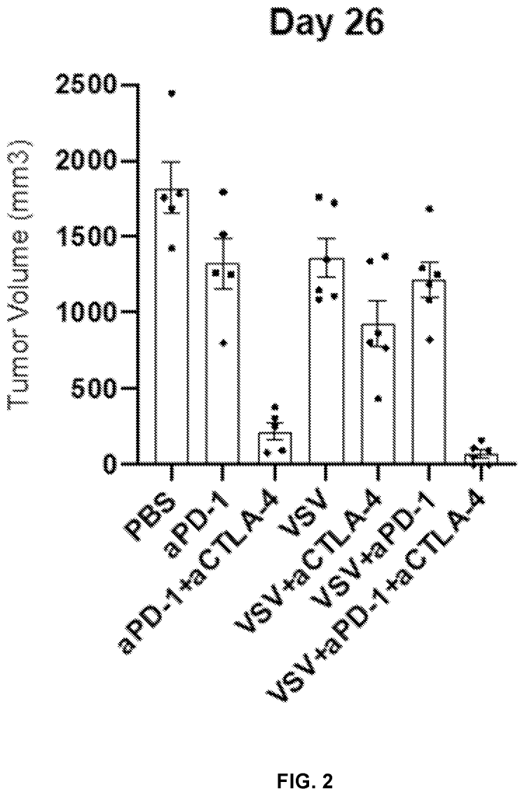

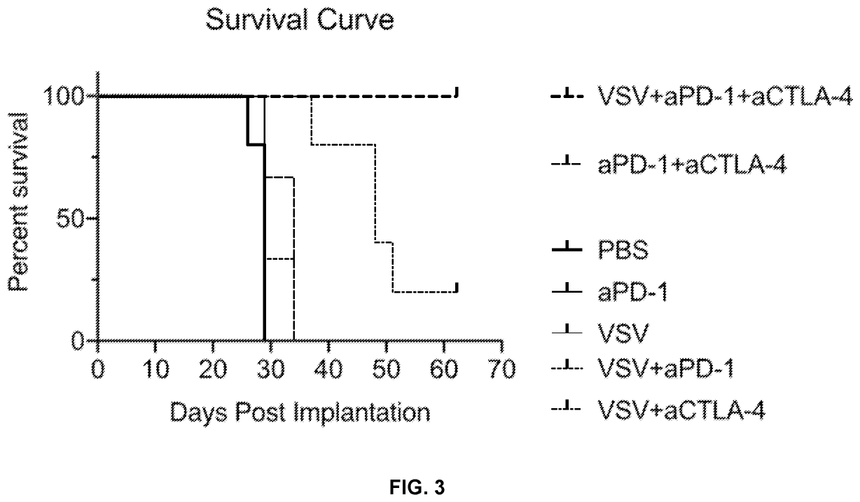

The average of tumor volumes over time for each group shows that monotherapy with either VSV or anti-PD-1 antibodies showed partial tumor growth inhibition compared to treatment with PBS and isotype control treated group (FIG. 1). Individual tumor volumes at day 26 after treatment initiation (FIG. 2) were used for statistical analysis, as this was the last time point in the study where all animals in all groups were alive. Statistical significance was determined by one-way ANOVA with Dunnett's multiple comparisons post-test (** p<0.01, **** p<0.0001). Monotherapy efficacy of anti-PD-1 antibody or VSV did not achieve statistical significance. Combination of VSV with anti-CTLA4 antibody or with anti-PD-1 antibodies treatment resulted in more efficacious tumor growth inhibition compared to monotherapy with anti-PD-1 antibody or control with statistically significant smaller tumors at day 26 in a combination treated group than in an anti-PD-1 antibody treated group (FIG. 3). Combination of anti-CTLA4 and anti-PD-1 antibodies treatment resulted in a statistically significant reduction in tumor growth compared to all the other mono- and dual-combinations without any tumor free mice by day 26. Of note, the triple combination VSV with anti-CTLA4 and anti-PD-1 antibodies treatment was more efficacious compared to all the other groups with all mice clearing their tumor by day 29, and this tumor clearance was durable until the end of the study by day 60 (FIGS. 1, 2, 3). Table 2 summarizes mean tumor volumes, percent survival, and numbers of tumor-free mice in each treatment group.

TABLE 2
Mean tumor volume, percent survival, and
numbers of tumor free mice in each treatment group

| | | Tumor | | | | |
|---|---|---|---|---|---|---|
| | | Volume, | | | | |
| Treat- | | mm³ | Tumor- | | | |
| ment | | mean | Free | Survival, % | | |
| group | | (±SD) | Mice | Day | Day | Day |
| (n = 5-6) | | Day 26 | Day 29 | 26 | 29 | 60 |
| 1 | PBS | 1821 (±168) | 0/5 | 100% | 0% | 0% |
| 2 | a-PD-1 | 1326 (±164) | 0/5 | 100% | 0% | 0% |
| 3 | a-PD-1 + | 223 (±59) | 1/5 | 100% | 100% | 20% |
| | a-CTLA4 | | | | | |
| 4 | VSV | 1364 (126) | 0/6 | 100% | 0% | 0% |
| 5 | VSV + a-PD-1 | 1220 (±115) | 0/6 | 100% | 16.6% | 0% |
| 6 | VSV + a-CTLA4 | 932 (±148) | 0/6 | 100% | 50% | 0% |
| 7 | VSV + a-PD-1 + | 70 (±26) | 6/6 | 100% | 100% | 100% |
| | a-CTLA4 | | | | | |

As shown in Table 2, mice treated with the triple combination VSV with anti-PD-1 and anti-CTLA4 antibodies were very efficacious at controlling and clearing large tumors during the course of the study, with six out of six mice being tumor free by day 29. Mice treated with either anti-PD-1 or anti-CTLA4 antibodies with or without combination with VSV exhibited a modestly reduced tumor volume as compared to controls at days 26 of the study. In contrast, treatment with the anti-PD1 and anti-CTLA4 antibodies dual combination demonstrated significant efficacy in reducing tumor volume in this study as compared to controls, with one mouse achieving tumor clearance out of five; but it was not as efficacious as when VSV was also delivered in combination to anti-PD1 and anti-CTLA4 antibodies. By day 29 of the study, all mice had to be eliminated in control PBS group, anti-PD-1 group, VSV-treated group, five out of six in the dual combination VSV with anti-PD-1 treated group, three out of six in the dual combination VSV with the anti-CTLA4 group, and all mice were still alive in anti-PD1 and anti-CTLA4 antibodies dual combination and the triple combination VSV with anti-PD-1 and anti-CTLA4 antibodies group. At the end of the study, only the triple combination group remained tumor free and survived, along with only one mouse out of five in the anti-PD1 and anti-CTLA4 antibodies dual combination group. No evidence of body weight loss was observed as a result of the triple combination therapy.

In summary, treatment with a combination of VSV with anti-mCTLA4 and anti-PD-1 antibodies resulted in reduced tumor growth and longer survival compared to monotherapy or dual therapy with either antibody and/or VSV.

### Example 2: Anti-Tumor Efficacy of the Triple Combination Anti-PD-1, Anti-CTLA4, and Oncolytic Virus VSV-M51R-GFP Delivered Intra-Tumor can be Achieved with Only One Dose of Anti-CTLA4 mlgG2a Antibody

This example describes the number of doses of the anti-CTLA4 antibody necessary to achieve anti-tumor efficacy in the triple combination using the oncolytic virus Vesicular Stomatitis Virus (VSV) with anti-PD-1 and anti-CTLA4 in wild type mice implanted with MC38 tumors. The VSV used in this study is a genetically attenuated virus named VSV-M51R-GFP as it encodes a mutation in the M protein (M51R) (M protein inhibits host cell protein production, but the M51R mutation preserves host cell protein production), and encodes for GFP, inserted between the G and L viral genes. C57BL/6 strain background mice from Jackson Laboratories were implanted subcutaneously with MC38 cells (3×10⁵ cells/mouse) at day 0. Tumors were measured using a caliper, and tumor volumes were calculated with the formula (L²×W)/2 where L is the smallest size. Mice were randomized evenly into 5 treatment groups when the average tumor size reached 150 mm³ which was at day 15. Mice were injected intratumorally with 50 μl of VSV-M51R-GFP virus at 5×10⁸ TCID₅₀ dose resuspended in PBS or PBS as control, and/or with intraperitoneal injection of 250 μg of either isotype control antibody and/or the anti-PD-1 antibody on days 15, 18, 22, and 25 and/or 250 μg of anti-CTLA4 antibody, with various doses amounts, either 4 doses (on days 15, 18, 22, and 25), or one dose (on days 15) or two doses (on days 15 and 18) (Table 3).

TABLE 3
Experimental dosing and
treatment protocol for groups of mice

| | | Virus | | | | | |
|---|---|---|---|---|---|---|---|
| | Virus | or | | | Ab 2 | Dosing | |
| | or | PBS | | Ab 1 | (route | interval | |
| | PBS | dosed | | (route | IP) | Anti- | n |
| Group | Route | at D15 | TCID₅₀ | IP) | # doses | bodies | # |
| 1 | Intra- | PBS | — | Isotype | Isotype | D15, | 7 |
| | tumor | | | rat | mIgG2a | 18, 22 | |
| | | | | IgG2a | 250 μg | and 25 | |
| | | | | 250 μg | | | |
| 2 | Intra- | PBS | — | a-PD-1 | a-CTLA4 | D15, | 7 |
| | tumor | | | 250 μg | mIgG2a | 18, 22 | |
| | | | | | 250 μg | and 25 | |
| | | | | | 4 doses | | |
| 3 | Intra- | VSV- | 5 × 10⁸ | a-PD-1 | a-CTLA4 | D15, | 7 |
| | tumor | M51R- | TCID₅₀ | 250 μg | mIgG2a | 18, 22 | |
| | | GFP | | | 250 μg | and 25 | |
| | | | | | 4 doses | | |
| 4 | Intra- | VSV- | 5 × 10⁸ | a-PD-1 | a-CTLA4 | D15, | 8 |
| | tumor | M51R- | TCID₅₀ | 250 μg | mIgG2a | 18, 22 | |
| | | GFP | | | 250 μg | and 25 | |
| | | | | | 1 dose | | |
| 5 | Intra- | VSV- | 5 × 10⁸ | a-PD-1 | a-CTLA4 | D15, | 8 |
| | tumor | M51R- | TCID₅₀ | 250 μg | mIgG2a | 18, 22 | |
| | | GFP | | | 250 μg | and 25 | |
| | | | | | 2 doses | | |

Tumor volumes were monitored by caliper measurements twice per week until the end of the study at day 60.

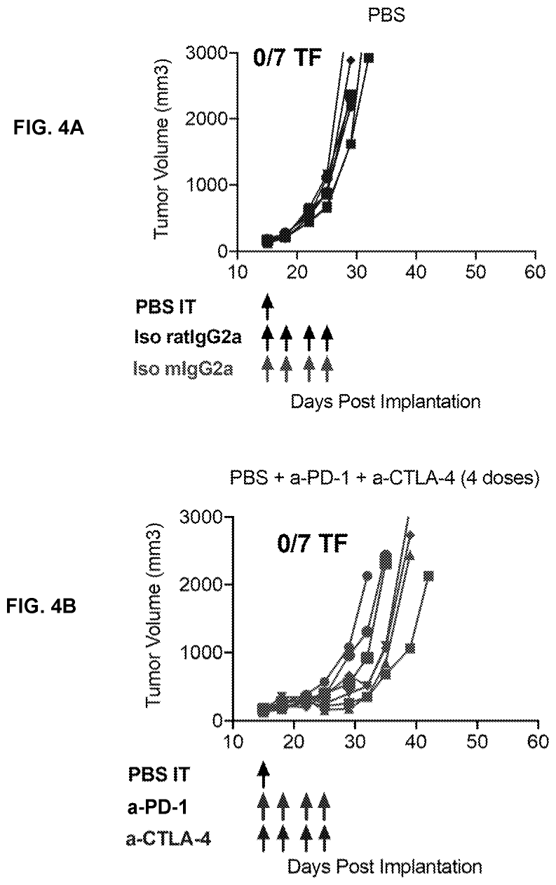

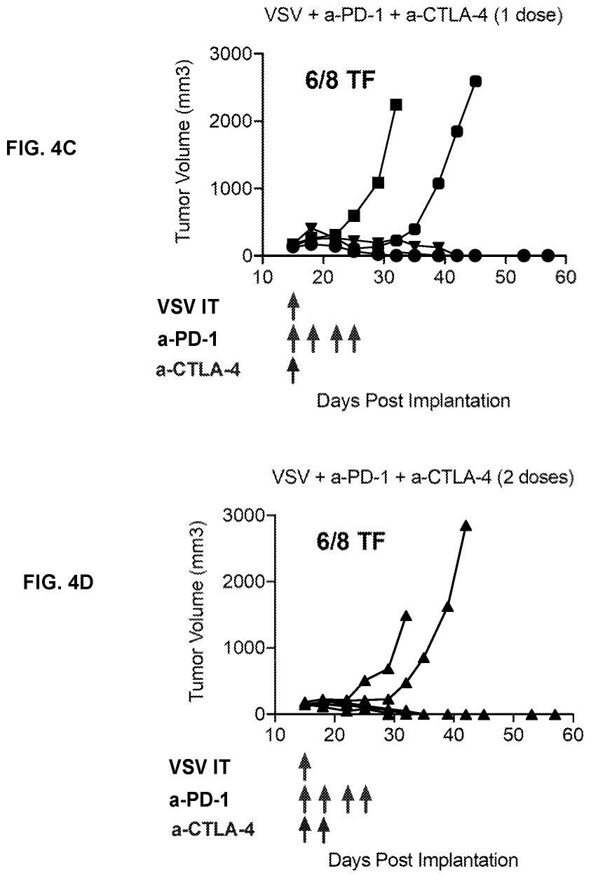

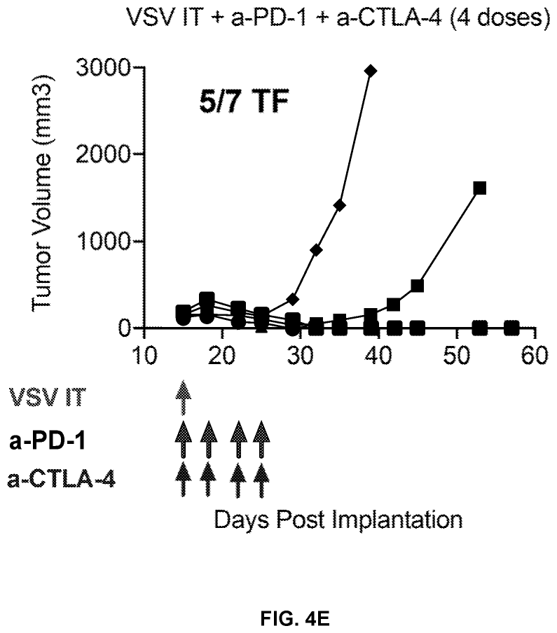

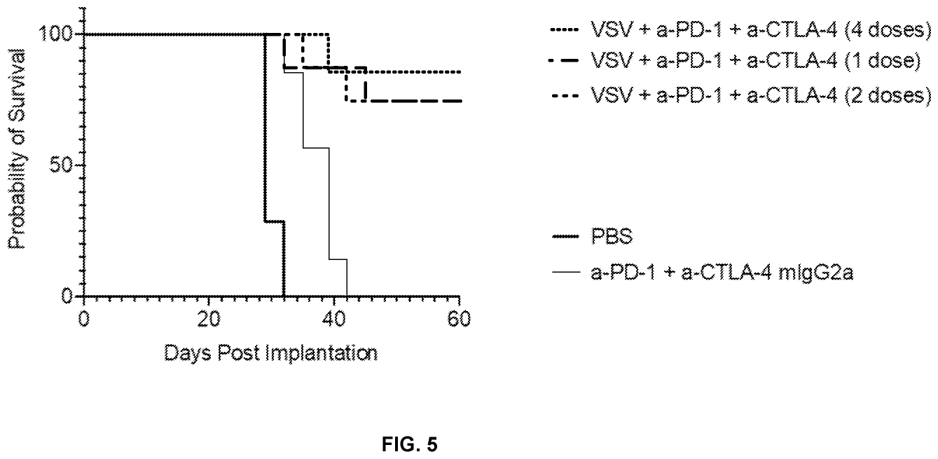

Table 4 summarizes mean tumor volumes, percent survival, and numbers of tumor-free mice in each treatment group. The anti-tumor efficacy of the triple combination VSV with anti-PD-1 antibody and anti-CTLA4 antibody in mlgG2a format was very similar in the groups receiving either one or two or four doses of the anti-CTLA4 antibody (FIGS. 4A, 4B, 4C, 4D, 4E), with six out of eight mice (75%) were tumor-free by day 45 in the one-dose and the two-doses groups compared to five out of seven mice (71%) in the 4-doses group. By day 60, the one-dose and two-doses groups had 62.5% of their mice surviving and tumor free compared to 71.4% of the 4-doses group (FIG. 5). These results suggest that the potent anti-tumor efficacy of the triple combination VSV with anti-PD-1 and anti-CTLA4 antibodies can be recapitulated with only one dose of anti-CTLA4 given simultaneously with the virus and the first dose of anti-PD-1 antibody.

TABLE 4
Mean tumor volume, percent survival, and numbers
of tumor free mice in each treatment group

| | | Tumor | | | | | |
|---|---|---|---|---|---|---|---|
| | | Volume, | Tumor- | | | | |
| | | mm³ | Free | | | | |
| | | mean | Mice | | Survival, % | | |
| Treatment group | | (±SD) | Day | Day | Day | Day | Day |
| (n = 5-6) | | Day 29 | 29 | 45 | 29 | 32 | 60 |
| 1 | PBS | 2404 (±294) | 0/7 | 0/7 | 100% | 0% | 0% |
| 2 | a-PD-1 + a-CTLA4 | 580 (±129) | 0/7 | 0/7 | 100% | 100% | 0% |
| | mIgG2a (4 doses) | | | | | | |
| 3 | VSV I.T. + a-PD-1 + | 70 (±45) | 3/7 | 5/7 | 100% | 100% | 71.4% |
| | a-CTLA4 mIgG2a | | | | | | |
| | (4 doses) | | | | | | |
| 4 | VSV I.T. + a-PD-1 + | 207 (±128) | 0/8 | 6/8 | 100% | 100% | 62.5% |
| | a-CTLA4 mIgG2a | | | | | | |
| | (1 dose) | | | | | | |
| 5 | VSV I.T. + a-PD-1 + | 140 (±82) | 3/7 | 6/8 | 100% | 100% | 62.5% |
| | a-CTLA4 mIgG2a | | | | | | |
| | (2 doses) | | | | | | |

### Example 3: Anti-Tumor Efficacy of the Triple Combination Anti-PD-1, Anti-CTLA4, and Oncolytic Virus VSV-M51R-GFP can be Achieved with Either Intra-Tumor or Intravenous Delivery of the Virus

This example describes the delivery route the virus can be delivered in in order to achieve anti-tumor efficacy in the triple combination using the oncolytic virus Vesicular Stomatitis Virus (VSV) with anti-PD-1 and anti-CTLA4 in wild type mice implanted with MC38 tumors. The VSV used in this example is a genetically attenuated virus named VSV-M51R-GFP as it encodes a mutation in the M protein (M51R) (M protein inhibits host cell protein production, but the M51R mutation preserves host cell protein production), and encodes for GFP, inserted between the G and L viral genes. C57BL/6 strain background mice from Jackson Laboratories were implanted subcutaneously with MC38 cells (3×10⁵ cells/mouse) at day 0. Tumors were measured using a caliper, and tumor volumes were calculated with the formula (L²×W)/2 where L is the smallest size. Mice were randomized evenly into 4 treatment groups when the average tumor size reached 150 mm³ which was at day 15. Mice were injected intratumorally with 50 μl of VSV-M51R-GFP virus at 5×10⁸ TCID₅₀ dose resuspended in PBS or 200 μl intravenous injection of VSV-M51R-GFP virus at 1×10⁹ TCID₅₀ dose resuspended in PBS, or PBS as control, and/or with intraperitoneal injection of 250 μg of either isotype control antibody and/or the anti-PD-1 antibody and/or 250 μg of the anti-CTLA4 antibody on days 15, 18, 22, and 25 (Table 5). Tumor volumes were monitored until the end of the study at day 60.

TABLE 5
Experimental dosing and treatment protocol for groups of mice

| | Virus | Virus | | | Ab 2 | Dosing | |
|---|---|---|---|---|---|---|---|
| | or | or PBS | | Ab 1 | (route | interval | |
| | PBS | dosed | | (route | IP) | Anti- | n |
| Group | Route | at D15 | TCID₅₀ | IP) | # doses | bodies | # |
| 1 | Intra- | PBS | — | Isotype | Isotype | D15, | 7 |
| | tumor | | | rat | mIgG2a | 18, 22 | |
| | | | | IgG2a | 250 μg | and 25 | |
| | | | | 250 μg | | | |
| 2 | Intra- | PBS | — | a-PD-1 | a-CTLA4 | D15, | 7 |
| | tumor | | | 250 μg | mIgG2a | 18, 22 | |
| | | | | | 250 μg | and 25 | |
| | | | | | 4 doses | | |
| 3 | Intra- | VSV- | 1 × 10⁹ | a-PD-1 | a-CTLA4 | D15, | 7 |
| | tumor | M51R- | TCID₅₀ | 250 μg | mIgG2a | 18, 22 | |
| | | GFP | | | 250 μg | and 25 | |
| | | | | | 4 doses | | |
| 4 | Intra- | VSV- | 1 × 10⁹ | a-PD-1 | a-CTLA4 | D15, | 8 |
| | venous | M51R- | TCID₅₀ | 250 μg | mIgG2a | 18, 22 | |
| | | GFP | | | 250 μg | and 25 | |
| | | | | | 4 doses | | |

Table 6 summarizes the mean tumor volume, percent survival, and number of tumor-free mice in each treatment group in this experiment.

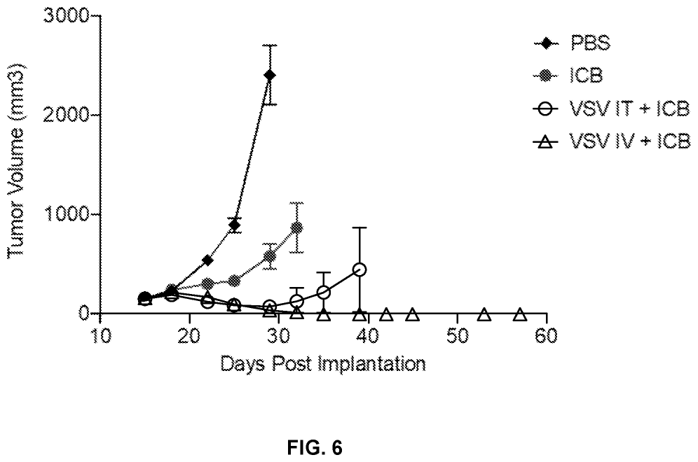

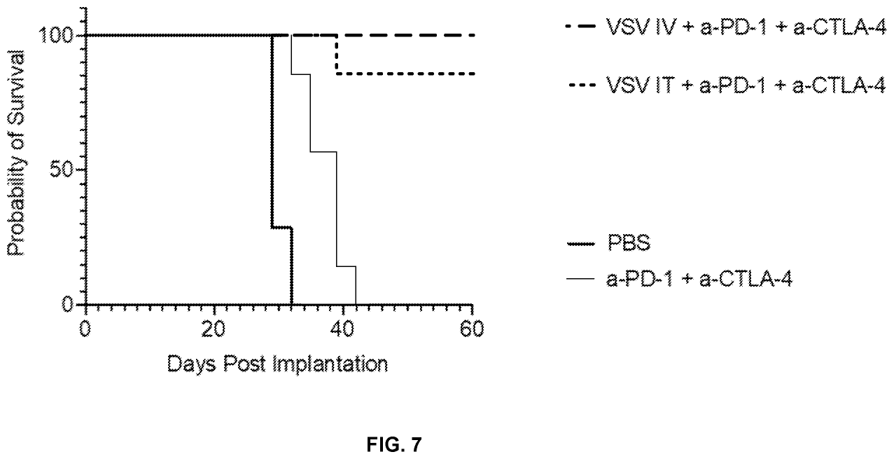

The anti-tumor efficacy of the triple combination VSV with anti-PD-1 and anti-CTLA4 antibodies was very robust in the groups receiving VSV either as a single dose intratumorally or as a single dose intravenously, with intravenous delivery trending to be more efficacious than intra-tumor (FIG. 6), with eight out of eight mice (100%) tumor-free by day 45 in the intravenous-dosed group compared to five out of seven mice (71%) in the intra-tumor group. By day 60, 100% of the intravenous-dosed group survived and were tumor free compared to 71.4% of the intra-tumor group (FIG. 7). The intravenous delivery of VSV robustly enhanced the anti-PD-1 and anti-CTLA4 combination checkpoint therapy and indicates the triple combination efficacy can be achieved with either intra-tumor or intravenous delivery of the virus.

TABLE 6
Mean tumor volume, percent survival and numbers of
tumor free mice in each treatment group

| | | Tumor | | | | | |
|---|---|---|---|---|---|---|---|
| | | Volume, | Tumor- | | | | |
| | | mm³ | Free | | | | |
| | | mean | Mice | | Survival, % | | |
| Treatment group | | (±SD) | Day | Day | Day | Day | Day |
| (n = 5-6) | | Day 29 | 29 | 45 | 29 | 32 | 60 |
| 1 | PBS | 2404 (±294) | 0/7 | 0/7 | 100% | 0% | 0% |
| 2 | a-PD-1 + a- | 580 (±129) | 0/7 | 0/7 | 100% | 100% | 0% |
| | CTLA 4mIgG2a | | | | | | |
| | (4 doses) | | | | | | |
| 3 | VSV I.T. + a-PD- | 70 (±45) | 3/7 | 5/7 | 100% | 100% | 71.4% |
| | 1 + a-CTLA4 | | | | | | |
| | mIgG2a | | | | | | |
| | (4 doses) | | | | | | |
| 4 | VSV I.V. + a- | 40 (±12) | 2/8 | 8/8 | 100% | 100% | 100% |
| | PD-1 + a-CTLA4 | | | | | | |
| | mIgG2a | | | | | | |
| | (4 doses) | | | | | | |

### Example 4: Anti-Tumor Efficacy of the Combination Treatment with Anti-PD-1, Anti-CTLA4 and Intravenous Delivery of Oncolytic Virus VSV-mlFNb-NIS in Mice Bearing 150 mm³ Average MC38 Tumors

This example describes the anti-tumor efficacy of a triple combination using the oncolytic virus Vesicular Stomatitis Virus (VSV) delivered intravenously with anti-PD-1 and anti-CTLA4 antibodies in wild type mice implanted with MC38 tumors. The VSV used in this study is a genetically attenuated virus VSV-mlFNb-NIS (or mVV1) that encodes for the mouse interferon-beta (IFNb), inserted between the M and G viral genes and for the sodium/iodide symporter (NIS) inserted between the G and L viral genes.

C57BL/6 strain background mice from Jackson Laboratories were implanted subcutaneously with MC38 cells (3×10⁵ cells/mouse) at day 0. Tumors were measured using a caliper and tumor volumes were calculated with the formula (L²×W)/2 where L is the smallest size. Mice were randomized evenly into eight treatment groups when the average tumor size reached 150 mm³ which was at day 15. Mice received an intravenous injection of 200 μl of mVV1 at 1×10⁹ TCID₅₀ dose resuspended in PBS or PBS as control, and/or with intraperitoneal injection of 250 μg of either isotype control antibody (mlgG2a and/or rat IgG2a) and/or 250 μg of the anti-CTLA4 antibody, and/or the anti-PD-1 antibody on days 15, 18, 21, and 24 (Table 7). Tumor volumes were monitored by caliper measurements twice per week until the end of the study at day 60.

TABLE 7
Experimental dosing and treatment protocol for groups of mice

| | | | | | Ab 1 | | Ab 2 | |
|---|---|---|---|---|---|---|---|---|
| | Virus | | | | dos- | | dos- | |
| | or | Virus | | Ab 1 | ing | Ab 2 | ing | |
| | PBS | or | | (route | inter- | (route | inter- | n |
| Group | Route | PBS | TCID₅₀ | IP) | val | IP) | val | # |
| 1 | Intra- | PBS | — | Isotype | D15, | Isotype | D15, | 7 |
| | venous | | | rat | 18, | mouse | 18, | |
| | | | | IgG2a | 21, | IgG2a | 21, | |
| | | | | 250 μg | 24 | 250 μg | 24 | |
| 2 | Intra- | PBS | — | a-PD-1 | D15, | Isotype | D15, | 7 |
| | venous | | | 250 μg | 18, | mouse | 18, | |
| | | | | | 21, | IgG2a | 21, | |
| | | | | | 24 | 250 μg | 24 | |
| 3 | Intra- | PBS | — | Isotype | D15, | a- | D15, | 7 |
| | venous | | | rat | 18, | CTLA4 | 18, | |
| | | | | IgG2a | 21, | mIgG2a | 21, | |
| | | | | 250 μg | 24 | 250 μg | 24 | |
| 4 | Intra- | PBS | — | a-PD-1 | D15, | a- | D15, | 7 |
| | venous | | | 250 μg | 18, | CTLA4 | 18, | |
| | | | | | 21, | mIgG2a | 21, | |
| | | | | | 24 | 250 μg | 24 | |
| 5 | Intra- | VSV- | 1 × 10⁹ | Isotype | D15, | Isotype | D15, | 7 |
| | venous | mIFNb- | TCID₅₀ | rat | 18, | mouse | 18, | |
| | | NIS | | IgG2a | 21, | IgG2a | 21, | |
| | | | | 250 μg | 24 | 250 μg | 24 | |
| 6 | Intra- | VSV- | 1 × 10⁹ | a-PD-1 | D15, | Isotype | D15, | 7 |
| | venous | mIFNb- | TCID₅₀ | 250 μg | 18, | mouse | 18, | |
| | | NIS | | | 21, | IgG2a | 21, | |
| | | | | | 24 | 250 μg | 24 | |
| 7 | Intra- | VSV- | 1 × 10⁹ | Isotype | D15, | a- | D15, | 7 |
| | venous | mIFNb- | TCID₅₀ | rat | 18, | CTLA4 | 18, | |
| | | NIS | | IgG2a | 21, | mIgG2a | 21, | |
| | | | | 250 μg | 24 | 250 μg | 24 | |
| 8 | Intra- | VSV- | 1 × 10⁹ | a-PD-1 | D15, | a- | D15, | 7 |
| | venous | mIFNb- | TCID₅₀ | 250 μg | 18, | CTLA4 | 18, | |
| | | NIS | | | 21, | mIgG2a | 21, | |
| | | | | | 24 | 250 μg | 24 | |

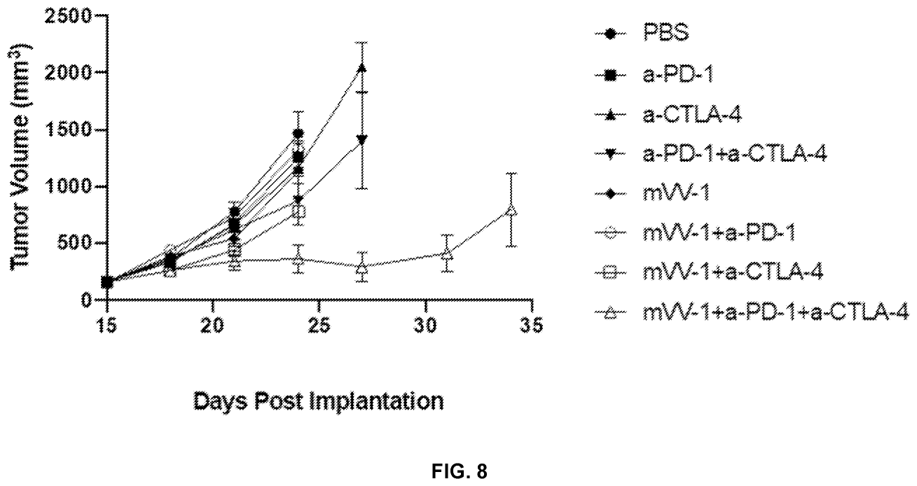

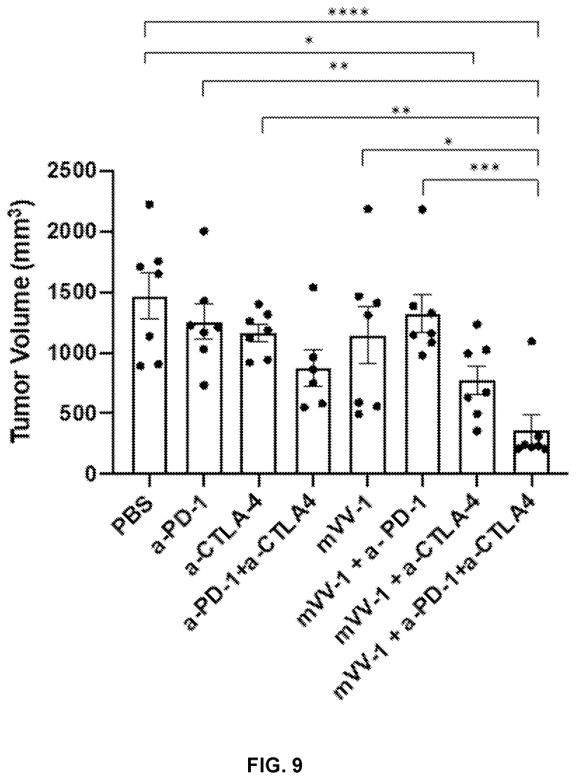

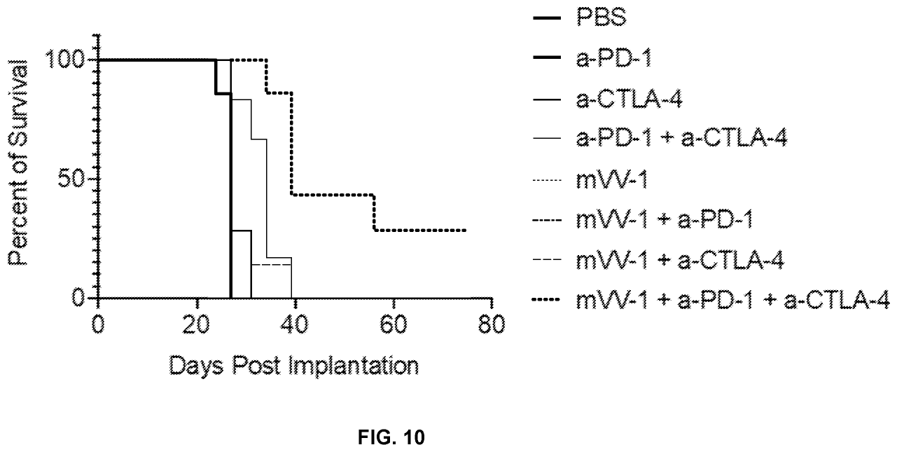

Table 8 summarizes the mean tumor volume, percent survival, and numbers of tumor-free mice in each treatment group. The average of tumor volumes over time for each group shows that monotherapy with either mVV1 or anti-PD-1 or anti-CTLA4 antibodies showed minor tumor growth inhibition compared to treatment with PBS and isotype control treated group (FIG. 8). Individual tumor volumes at day 24 after treatment initiation (FIG. 9) were used for statistical analysis, as this was the last time point in the study where all animals in all groups were alive. Statistical significance was determined by one-way ANOVA with Dunnett's multiple comparisons post-test (** p<0.01, **** p<0.0001). Monotherapy of anti-PD-1 or anti-CTLA4 antibodies or mVV1 did not achieve statistical significance, neither did the combination of mVV1 with anti-PD-1 antibody. The combination of mVV1 to anti-CTLA4 antibody treatment resulted in more efficacious tumor growth inhibition compared to monotherapy with anti-PD-1 or anti-CTLA4 antibodies or mVV1 or control (FIG. 10). The combination of anti-CTLA4 and anti-PD-1 antibodies resulted in reduced tumor growth, but it did not result in a statistically significant reduction at day 24 compared to all the other mono- and dual-combinations. Of note, the triple combination of intravenously delivered mVV1 with anti-CTLA4 and anti-PD-1 antibodies treatment was highly efficacious compared to all the other groups with two out of seven mice remaining tumor free until the end of the study by day 60 (FIGS. 8, 9, 10). No evidence of body weight loss was observed as a result of the triple combination therapy. In summary, treatment with a combination of intravenously delivered mVV1 with anti-mCTLA4 and anti-PD-1 antibodies resulted in reduced tumor growth and improved survival compared to monotherapy or dual therapy with either antibody and/or mVV1.

TABLE 8
Mean tumor volume, percent survival, and numbers
of tumor free mice in each treatment group

| | | Tumor | | | | | |
|---|---|---|---|---|---|---|---|
| | | Volume, | Tumor- | | | | |
| | | mm³ | Free | | | | |
| Treatment | | mean | Mice | | Survival, % | | |
| group | | (±SD) | Day | Day | Day | Day | Day |
| (n = 5-6) | | Day 24 | 24 | 42 | 29 | 42 | 60 |
| 1 | PBS | 1468 (±189) | 0/7 | 0/7 | 100% | 0% | 0% |
| 2 | a-PD-1 | 1257 (±148) | 0/7 | 0/7 | 100% | 0% | 0% |
| 3 | a-CTLA4 | 1164 (±69) | 0/7 | 0/7 | 100% | 0% | 0% |
| 4 | a-PD-1 + | 875 (±148) | 0/7 | 0/7 | 100% | 0% | 0% |
| | a-CTLA4 | | | | | | |
| 5 | mVV1 I.V. | 1146 (±237) | 0/7 | 0/7 | 100% | 0% | 0% |
| 6 | mVV1 I.V. + | 1324 (±152) | 0/7 | 0/7 | 100% | 0% | 0% |
| | a-PD-1 | | | | | | |
| 7 | mVV1 I.V. + | 773 (±120) | 0/7 | 0/7 | 100% | 0% | 0% |
| | a-CTLA4 | | | | | | |
| 8 | mVV1 I.V. + a- | 361 (±123) | 0/7 | 2/7 | 100% | 42.9% | 28.6% |
| | PD-1 + a-CTLA4 | | | | | | |

### Example 5: Anti-Tumor Efficacy of the Triple Combination Anti-PD-1, Anti-CTLA4 and Oncolytic Virus VSV-M51R-GFP Delivered Intra-Tumor can be Achieved with Lower Dose of Anti-CTLA4 mlgG2a Antibody

This example describes the reduced dose of the anti-CTLA4 antibody that still achieves anti-tumor efficacy in the triple combination using the oncolytic virus Vesicular Stomatitis Virus (VSV) with anti-PD-1 and anti-CTLA4 in wild type mice implanted with MC38 tumors. The VSV used in this study is a genetically attenuated virus named VSV-M51R-GFP as it encodes a mutation in the M protein (M51R) (M protein inhibits host cell protein production, but the M51R mutation preserves protein production), and encodes for GFP, inserted between the G and L viral genes. The anti-PD-1 antibody used in this study is the clone 29F1.A12 rat IgG2a from Bioxcell, the anti-CTLA4 antibodies used is clone 9D9 in mlgG2a format purchased from Invivogen. C57BL/6 strain background mice from Jackson Laboratories were implanted subcutaneously with MC38 cells (3×105 cells/mouse) at day 0. Tumors were measured using a caliper and tumor volumes were calculated with the formula (L2×W)/2 where L is the smallest size. Mice were randomized evenly into four treatment groups when the average tumor size reached 150 mm³ which was at day 15. Mice were injected intratumorally with 50 μl of VSV-M51R-GFP virus at 5×108 TCID50 dose resuspended in PBS or PBS as control, and/or with intraperitoneal injection of 250 μg of either isotype control antibody and/or anti-mouse PD-1 rat IgG2a antibody (29F1.A12) on days 15, 18, 22 and 25 and/or 250 μg or 50 μg of anti-mouse CTLA4-mlgG2a (clone 9D9), at 4 doses (on days 15, 18, 22 and 25). Tumor volumes were monitored by caliper measurements twice per week until the end of the study at day 60.

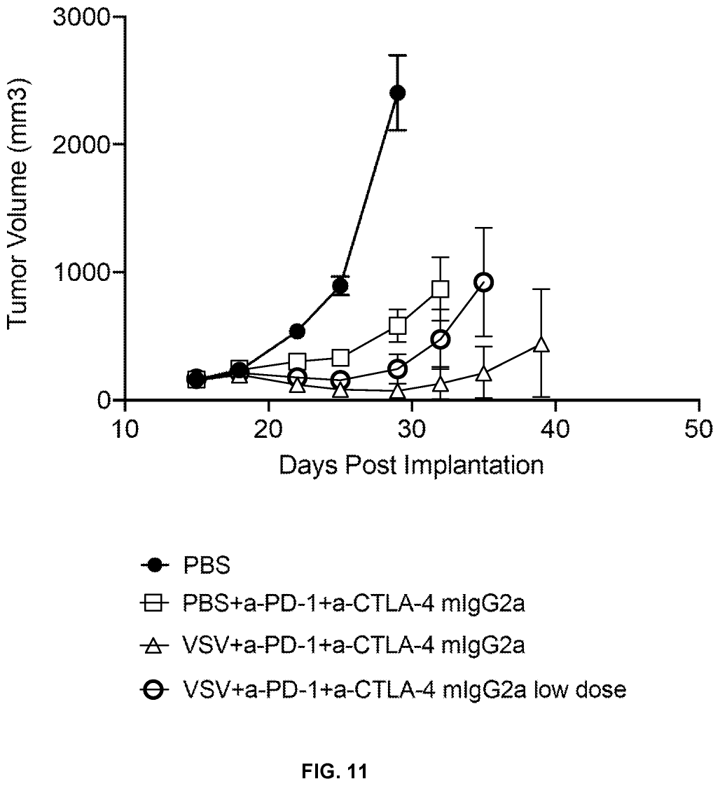

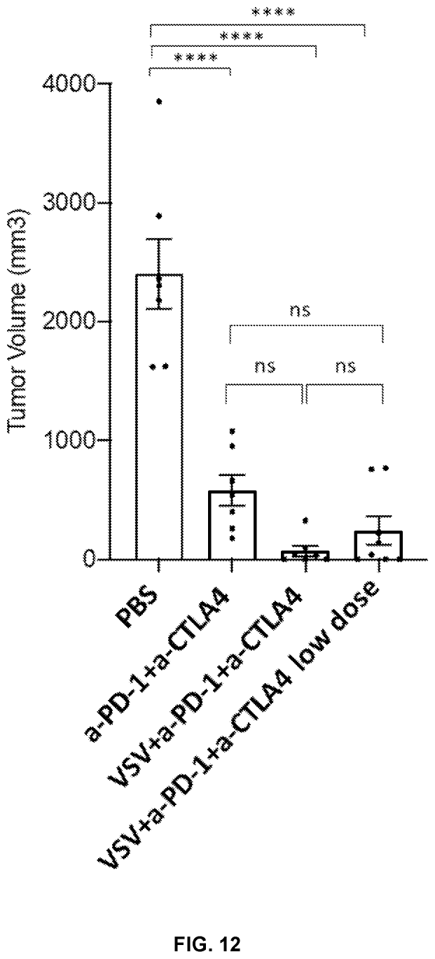

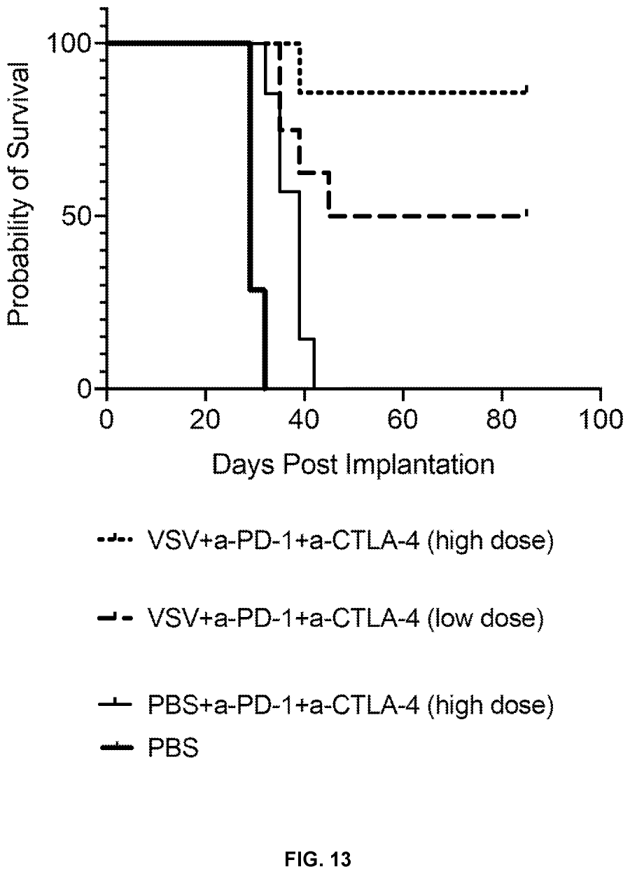

The anti-tumor efficacy of the triple combination VSV with anti-PD-1 antibody and anti-CTLA4 antibody was observed in the group receiving a lower dose of the anti-CTLA4 antibody (FIGS. 11, 12), with four out of eight mice (50%) being tumor-free by day 45 compared to five out of seven mice (71%) in the higher dose group. By day 60, the lower-dose group had 50% of their mice surviving and tumor free compared to 71.4% of the 4-doses group (FIG. 13). These results suggest that the amount of anti-CTLA4 administered in the triple combination VSV with anti-PD-1 and anti-CTLA4 antibodies can be reduced to still achieve anti-tumor efficacy.

TABLE 9
Mean tumor volume, percent survival and numbers of tumor free
mice in each treatment group from in vivo tumor

| | | Tumor | | | | | |
|---|---|---|---|---|---|---|---|
| | | Volume, | Tumor- | | | | |
| | | mm³ | Free | | | | |
| Treatment | | mean | Mice | | Survival, % | | |
| group | | (±SD) | Day | Day | Day | Day | Day |
| (n = 5-6) | | Day 29 | 29 | 45 | 29 | 32 | 60 |
| 1 | PBS | 2404 (±294) | 0/7 | 0/7 | 100% | 0% | 0% |
| 2 | a-PD-1 + a-CTLA4 | 580 (±129) | 0/7 | 0/7 | 100% | 100% | 0% |
| | mIgG2a (high dose) | | | | | | |
| 3 | VSV LT. + a-PD-1 + | 70 (±45) | 3/7 | 5/7 | 100% | 100% | 71.4% |
| | a-CTLA4 mIgG2a | | | | | | |
| | (high dose) | | | | | | |
| 4 | VSV LT. + a-PD-1 + | 240 (±117) | 0/8 | 4/8 | 100% | 100% | 50% |
| | a-CTLA4 mIgG2a | | | | | | |
| | (low dose) | | | | | | |

As shown in Table 9, mice treated with the triple combination VSV I.T. (intratumor) with anti-PD-1 and anti-CTLA4 antibodies was very effective at controlling tumor growth, with five out of seven mice being tumor free by day 45. Reducing the anti-CTLA4 doses by five-fold (from 250 μg to 50 μg) showed surprisingly significant anti-tumor efficacy when combined to VSV with anti-PD-1 antibody.

### Example 6: Anti-Tumor Efficacy of the Combination Treatment with Anti-PD-1, One Dose of Anti-CTLA4 with Intravenous Delivery of Oncolytic Virus VSV-mlFNb-NIS in Mice Bearing 150 mm³ Average MC38 Tumors

This example describes the anti-tumor efficacy of a triple combination using the oncolytic virus Vesicular Stomatitis Virus (VSV) encoding IFNb and NIS with anti-PD-1 and one dose anti-CTLA4 in wild type mice implanted with MC38 tumors. The VSV used in this study is a genetically attenuated virus named VSV-mlFNb-NIS (or mVV1) as it encodes for the mouse interferon beta (IFNb), inserted between the M and G viral genes and for the sodium/iodide symporter (NIS) inserted between the G and L viral genes. The anti-PD-1 antibody used in this study is the clone 29F1.A12 rat IgG2a from Bioxcell, and the anti-CTLA4 antibody used was clone 9D9 in mlgG2a format from Invivogen.

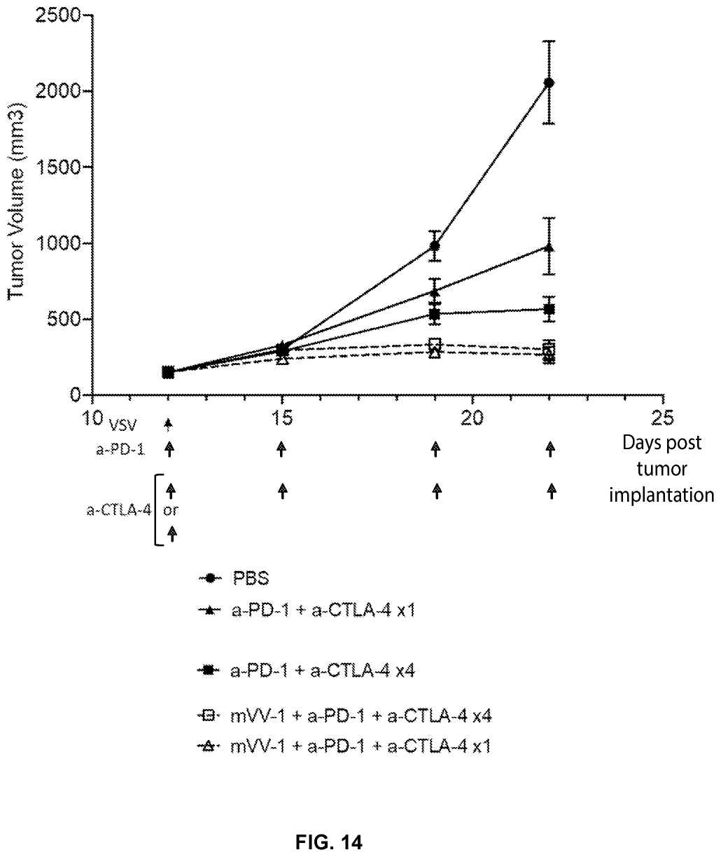

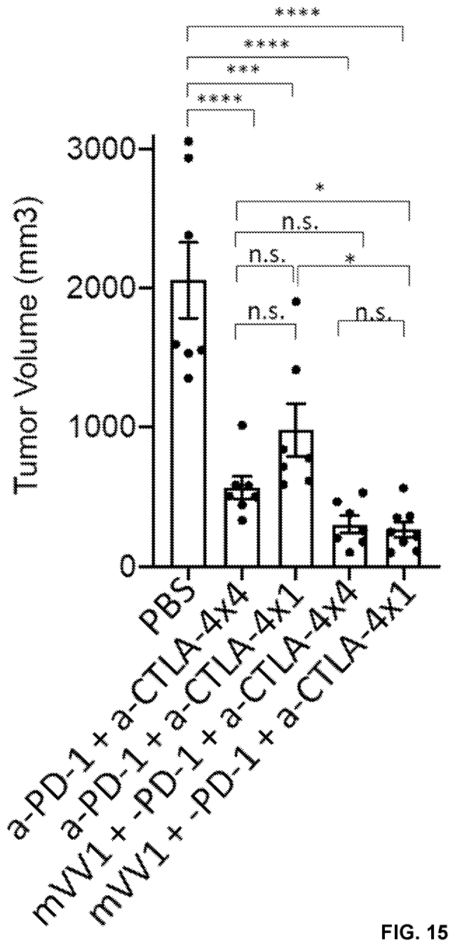

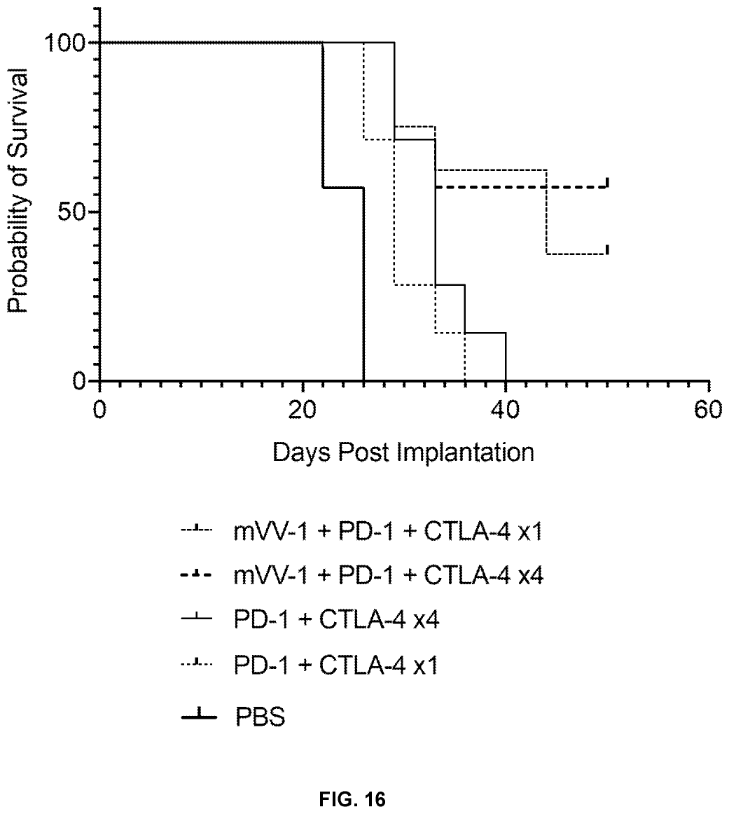

C57BL/6 strain background mice from Jackson Laboratories were implanted subcutaneously with MC38 cells (3×10⁵ cells/mouse) at day 0. Tumors were measured using a caliper, and tumor volumes were calculated with the formula (L²×W)/2 where L is the smallest size. Mice were randomized evenly into treatment groups when the average tumor size reached 150 mm³ which was at day 12. Mice received an intravenous injection of 200 μl of mVV1 at 1×10⁹ TCID50 dose resuspended in PBS or PBS as control, and/or with an intraperitoneal injection of 250 μg of either isotype control antibody (mlgG2a and/or rat IgG2a) and/or anti-mouse PD-1 rat IgG2a antibody (29F1.A12) on days 12, 15, 19, and 22 and/or 250 μg of anti-mouse CTLA4-mlgG2a antibody (clone 9D9) as a single dose on day 12 or 15 or four doses on days 12, 15, 19, and 22. Tumor volumes were monitored by caliper measurements twice per week until the end of the study at day 60. The average of tumor volumes over time for each group shows that one dose of anti-CTLA4 antibodies showed similar tumor growth inhibition compared to treatment with four doses of anti-CTLA4 when administered in combination with mVV1 and anti-PD-1 (FIG. 14). Individual tumor volumes at day 22 after treatment initiation (FIG. 15) were used for statistical analysis, as this was the last time point in the study where all animals in all groups were alive. Statistical significance was determined by one-way ANOVA with Dunnett's multiple comparisons post-test (** p<0.01, **** p<0.0001). The combination of mVV1 with one dose of anti-CTLA4 antibody treatment resulted in equivalent efficacy in tumor growth inhibition compared to four doses of anti-CTLA4 antibodies (FIGS. 14, 15). Notably, the triple combination of intravenously delivered mVV1 with one dose of anti-CTLA4 and anti-PD-1 antibodies treatment also led to increased survival, similar to the group that received four doses of CTLA4 (FIG. 16). In summary, treatment with a combination of intravenously delivered mVV1 with anti-PD-1 and one dose of anti-mCTLA4 antibodies resulted in similar efficacy to administering four doses of anti-mCTLA4 antibodies.

TABLE 10
Experimental dosing and treatment protocol for groups of mice

| | Virus | | | | | | | | | |
|---|---|---|---|---|---|---|---|---|---|---|
| | or | Virus | | Ab l | Ab l | | Ab 2 | Ab 3 | Ab 3 | |
| | PBS | or | | (route | dosing | Ab 2 | dosing | (route | dosing | n |
| Group | Route | PBS | TCID₅₀ | IP) | interval | (route IP) | interval | IP) | interval | # |
| 1 | Intra- | PBS | — | Isotype | D12, 15, | | | Isotype | D12, 15, | 7 |
| | venous | | | rat | 19, 22 | | | mouse | 19, 22 | |
| | | | | IgG2a | | | | IgG2a | | |
| | | | | 250 μg | | | | 250 μg | | |
| 2 | Intra- | PBS | — | a-PD-1 | D12, 15, | a-CTLA4 | D12, 15, | | | 7 |
| | venous | | | 250 μg | 19, 22 | mIgG2a | 19, 22 | | | |
| | | | | | | 250 μg | | | | |
| 3 | Intra- | PBS | — | a-PD-1 | D12, 15, | a-CTLA4 | D12 | Isotype | D15, 19, | 7 |
| | venous | | | 250 μg | 19, 22 | mIgG2a | | mouse | 22 | |
| | | | | | | 250 μg | | IgG2a | | |
| | | | | | | | | 250 μg | | |
| 4 | Intra- | mVV1 | 1 × 10⁹ | a-PD-1 | D12, 15, | a-CTLA4 | D12, 15, | | | 7 |
| | venous | | TCID⁵⁰ | 250 μg | 19, 22 | mIgG2a | 19, 22 | | | |
| | | | | | | 250 μg | | | | |
| 5 | Intra- | mVV1 | 1 × 10⁹ | a-PD-1 | D12, 15, | a-CTLA4 | D12 | Isotype | D15, 19, | 8 |
| | venous | | TCID⁵⁰ | 250 μg | 19, 22 | mIgG2a | | mouse | 22 | |
| | | | | | | 250 μg | | IgG2a | | |
| | | | | | | | | 250 μg | | |
| 6 | Intra- | mVV1 | 1 × 10⁹ | a-PD-1 | D12, 15, | a-CTLA4 | D15 | Isotype | D12, 19, | 8 |
| | venous | | TCID⁵⁰ | 250 μg | 19, 22 | mIgG2a | | mouse | 22 | |
| | | | | | | 250 μg | | IgG2a | | |
| | | | | | | | | 250 μg | | |

TABLE 11
Mean tumor volume, percent survival, and numbers of tumor
free mice in each treatment group from in vivo tumor

| | | Tumor | | | | |
|---|---|---|---|---|---|---|
| | | Volume, | Tumor- | | | |
| | | mm³ | Free | | | |
| Treatment | | mean | Mice | Survival, % | | |
| group | | (±SD) | Day | Day | Day | Day |
| (n = 5-6) | | Day 22 | 29 | 22 | 29 | 48 |
| 1 | PBS | 2057 (±719) | 0/7 | 100% | 0% | 0% |
| 2 | a-PD-1 + a-CTLA4 | 566 (±216) | 0/7 | 100% | 85% | 0% |
| | D12, 15, 19, 22 | | | | | |
| | a-PD-1 + a-CTLA4 | 980 (±492) | 0/7 | 100% | 71% | 0% |
| | D12 | | | | | |
| 3 | mVV1 + a-PD-1 + | 303 (±158) | 0/7 | 100% | 100% | 57% |
| | a-CTLA4 D12, | | | | | |
| | 15, 19, 22 | | | | | |
| 4 | mVV1 + a-PD-1 + | 266 (155) | 2/8 | 100% | 100% | 37.5% |
| | a-CTLA4 D12 | | | | | |
| 5 | mVV1 + a-PD-1 + | 1130 (±565) | 0/8 | 100% | 37.5% | 0% |
| | a-CTLA4 D15 | | | | | |

As shown in Table 11, mice treated with the triple combination mVV1 with anti-PD-1 and four doses of anti-CTLA4 antibodies were very efficacious at controlling and clearing large tumors during the course of the study. Mice treated with triple combination mVV1 with anti-PD-1 and one dose of anti-CTLA4 antibody given concomitantly with the virus exhibited similar reduced tumor volume compared to four doses. In contrast, if the one dose anti-CTLA4 antibody was given three days post the virus with anti-PD-1, the efficacy of the triple combination was abrogated. This data indicates that one dose of anti-CTLA4 given concomitantly to mVV1 and continuous dosing of anti-PD-1 can be used to achieve strong anti-tumor efficacy.

### Example 7: Anti-Tumor Efficacy of the Combination Treatment with Anti-PD-1, One Dose of Anti-CTLA4 Administered Concomitantly to Intravenous Delivery of Oncolytic Virus VSV-mlFNb-NIS in Mice Bearing 150 mm³ Average MC38 Tumors

This example describes the anti-tumor efficacy of a triple combination using the oncolytic virus Vesicular Stomatitis Virus (VSV) encoding IFNb and NIS with anti-PD-1 and one dose of anti-CTLA4 in wild type mice implanted with MC38 tumors. The VSV used in this study is a genetically attenuated virus named VSV-mlFNb-NIS (or mVV1) as it encodes for the mouse interferon beta (IFNb), inserted between the M and G viral genes and for the sodium/iodide symporter (NIS) inserted between the G and L viral genes. The anti-PD-1 antibody used in this study is the clone 29F1.A12 rat IgG2a from Bioxcell, and the anti-CTLA4 antibody used was clone 9D9 in mlgG2a format from Invivogen.

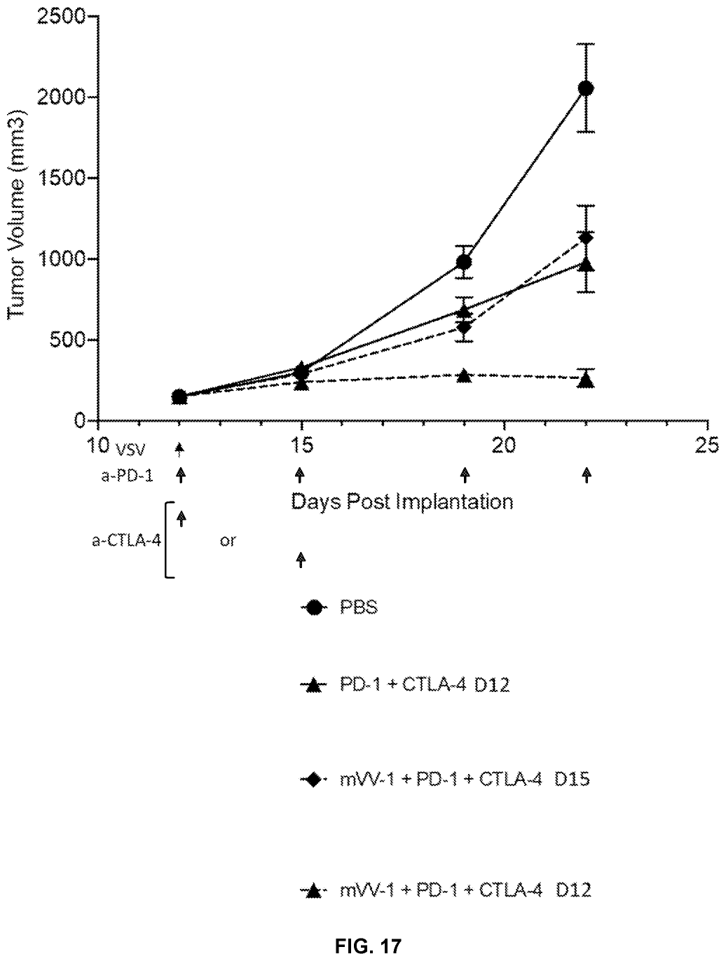

C57BL/6 strain background mice from Jackson Laboratories were implanted subcutaneously with MC38 cells (3×10⁵ cells/mouse) at day 0. Tumors were measured using a caliper, and tumor volumes were calculated with the formula (L²×W)/2 where L is the smallest size. Mice were randomized evenly into four treatment groups when the average tumor size reached 150 mm³ which was at day 12. Mice received an intravenous injection of 200 μl of mVV1 at 1×10⁹ TCID50 dose resuspended in PBS or PBS as control, and/or with intraperitoneal injection of 250 μg of either isotype control antibody (mlgG2a and/or rat IgG2a) and/or anti-mouse PD-1 rat IgG2a antibody (29F1.A12) on days 12, 15, 19, and 22 and/or 250 μg of anti-mouse CTLA4-mlgG2a antibody (clone 9D9) as a single dose on day 12 or 15. Tumor volumes were monitored by caliper measurements twice per week until the end of the study at day 60. The average of tumor volumes over time for each group shows that the one dose of anti-CTLA4 antibodies needs to be administered concomitantly to the virus, as the group that received a-CTLA4 at day 15 had reduced anti-tumor control compared to the group received it at day 12 (FIG. 17).

In summary, treatment with a combination of intravenously delivered mVV1 with anti-PD-1 and one dose of anti-mCTLA4 antibodies administered simultaneously to the virus resulted in strong anti-tumor efficacy.

### Example 8: Anti-Tumor Efficacy of the Combination Treatment with Anti-PD-1, One Dose of Anti-CTLA4 with Intravenous Delivery of Oncolytic Virus VSV-mlFNb-NIS in Mice Bearing 100 mm³ Average B16F10 Tumors

This example describes the anti-tumor efficacy of a triple combination using the oncolytic virus Vesicular Stomatitis Virus (VSV) encoding IFNb and NIS with anti-PD-1 and one dose of anti-CTLA4 in wild type mice implanted with B16F10 tumors. The VSV used in this study is a genetically attenuated virus named VSV-mlFNb-NIS (or mVV1) as it encodes for the mouse interferon beta (IFNb), inserted between the M and G viral genes and for the sodium/iodide symporter (NIS) inserted between the G and L viral genes. The anti-PD-1 antibody used in this study is the clone 29F1.A12 rat IgG2a from Bioxcell, and the anti-CTLA4 antibody used was clone 9D9 in mlgG2a format from Invivogen.

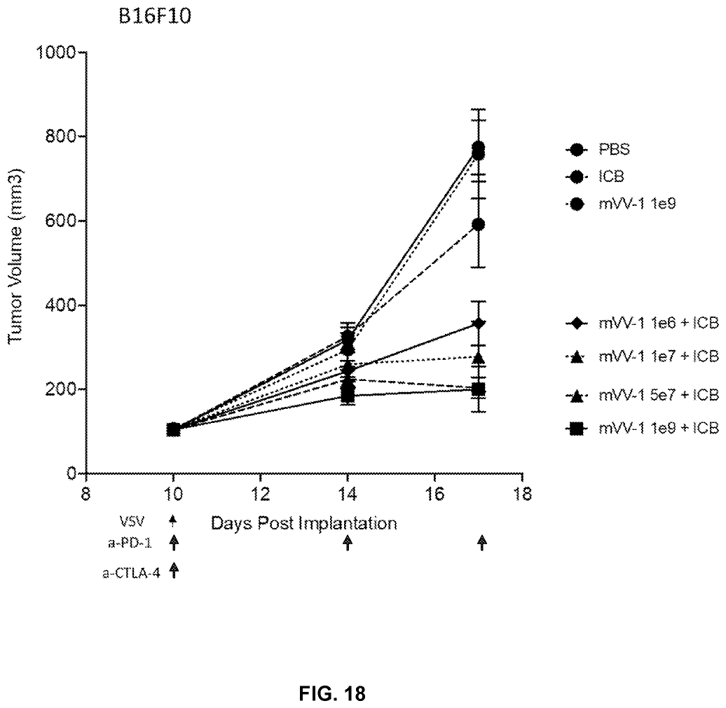

C57BL/6 strain background mice from Jackson Laboratories were implanted subcutaneously with B16F10 cells (5×10⁵ cells/mouse) at day 0. Tumors were measured using a caliper, and tumor volumes were calculated with the formula (L²×W)/2 where L is the smallest size. Mice were randomized evenly into seven treatment groups when the average tumor size reached 100 mm³ which was at day 10. Mice received an intravenous injection of 200 μl of mVV1 at either 1×10⁹ or 5×10⁷ or 1×10⁷ or 1×10⁶ TCID50 dose resuspended in PBS or PBS as control, and/or with intraperitoneal injection of 250 μg of either isotype control antibody (mlgG2a and/or rat IgG2a) and/or anti-mouse PD-1 rat IgG2a antibody (29F1.A12) on days 12, 15, 19 and 22 and/or 250 μg of anti-mouse CTLA4-mlgG2a antibody (clone 9D9) as a single dose on day 10. Tumor volumes were monitored by caliper measurements twice per week until the end of the study at day 60. The average of tumor volumes over time for each group shows that one dose of anti-CTLA4 antibodies showed strong tumor growth inhibition in B16F10 model compared to PBS or single agent (FIG. 18). Notably, the triple combination of intravenously delivered mVV1 with one dose anti-CTLA4 and anti-PD-1 antibodies treatment also led to increased survival similar efficacy when the virus dose was lowered from 1×10⁹ to 5×10⁷ or 1×10⁷ or 1×10⁶ TCID50 (FIG. 18).

In summary, treatment with a combination of intravenously delivered mVV1 with anti-PD-1 and one dose of anti-mCTLA4 antibodies resulted in strong anti-tumor efficacy in the B16F10 ant-PD-1 resistant tumor model, and a large range of the virus titer can still achieve strong combination efficacy.

TABLE 12
Experimental dosing and treatment protocol for groups of mice

| | Virus | | | | | | Ab 2 | |
|---|---|---|---|---|---|---|---|---|
| | or | Virus | | Ab 1 | Ab l | Ab 2 | dosing | |
| | PBS | or | | (route | dosing | (route | inter- | n |
| Group | Route | PBS | TCID₅₀ | IP) | interval | IP) | val | # |
| 1 | Intra- | PBS | — | Isotype | D10, 14, | Isotype | D10 | 5 |
| | venous | | | rat | 17, 21 | mouse | | |
| | | | | IgG2a | | IgG2a | | |
| | | | | 250 μg | | 250 μg | | |
| 2 | Intra- | PBS | — | a-PD-1 | D10, 14, | a- | D10 | 6 |
| | venous | | | 250 μg | 17, 21, | CTLA4 | | |
| | | | | | 24, 28 | mIgG2a | | |
| | | | | | | 250 μg | | |
| 3 | Intra- | mVV1 | 1 × 10⁹ | Isotype | D10, 14, | Isotype | D10 | 6 |
| | venous | | TCID⁵⁰ | rat | 17, 21 | mouse | | |
| | | | | IgG2a | | IgG2a | | |
| | | | | 250 μg | | 250 μg | | |
| 4 | Intra- | mVV1 | 1 × 10⁹ | a-PD-1 | D10, 14, | a- | D10 | 7 |
| | venous | | TCID⁵⁰ | 250 μg | 17, 21, | CTLA4 | | |
| | | | | | 24, 28 | mIgG2a | | |
| | | | | | | 250 μg | | |
| 5 | Intra- | mVV1 | 5 × 10⁷ | a-PD-1 | D10, 14, | a- | D10 | 7 |
| | venous | | TCID⁵⁰ | 250 μg | 17, 21, | CTLA4 | | |
| | | | | | 24, 28 | mIgG2a | | |
| | | | | | | 250 μg | | |
| 6 | Intra- | mVV1 | 1 × 10⁷ | a-PD-1 | D10, 14, | a- | D10 | 6 |
| | venous | | TCID⁵⁰ | 250 μg | 17, 21, | CTLA4 | | |
| | | | | | 24, 28 | mIgG2a | | |
| | | | | | | 250 μg | | |
| 7 | Intra- | mVV1 | 1 × 10⁶ | a-PD-1 | D10, 14, | a- | D10 | 7 |
| | venous | | TCID⁵⁰ | 250 μg | 17, 21, | CTLA4 | | |
| | | | | | 24, 28 | mIgG2a | | |
| | | | | | | 250 μg | | |

TABLE 13
Mean tumor volume, percent survival, and numbers of tumor free mice in each
treatment group from in vivo tumor using the B16F10 melanoma tumor model.

| | | Tumor Volume, | | | | |
|---|---|---|---|---|---|---|
| | | mm³ mean (±SD) | | Survival, % | | |
| Treatment group (n = 5-6) | | Day 17 | | Day 17 | Day 24 | Day 28 |
| 1 | PBS | 774 | (±143) | 100% | 80% | 0% |
| 2 | mVV1 1e9 TCID50 | 758 | (±259) | 100% | 66% | 0% |
| 3 | a-PD-1 + a-CTLA4 | 592 | (±249) | 100% | 66% | 16.6% |
| 4 | mVV1 1e9 TCID50 + a-PD-1 + a-CTLA4 | 200 | (±142) | 100% | 100% | 71% |
| 5 | mVV1 5e7 TCID50 + a-PD-1 + a-CTLA4 | 203 | (±65) | 100% | 100% | 100% |
| 6 | mVV1 1e7 TCID50 + a-PD-1 + a-CTLA4 | 277 | (±66) | 100% | 100% | 100% |
| 7 | mVV1 1e6 TCID50 + a-PD-1 + a-CTLA4 | 356 | (±138) | 100% | 100% | 85% |

As shown in Table 13, using the B16F10 subcutaneous tumor model, mice treated with either mVV1 or with anti-PD-1 combined with one dose anti-CTLA4 antibodies had very modest effect on the tumor growth in this high bar immune checkpoint resistant tumor model. However, the triple combination mVV1 with anti-PD-1 combined with one dose of anti-CTLA4 antibodies substantially added anti-tumor efficacy compared to the other groups. The data indicate that VSV can render checkpoint-resistant tumors sensitive to immunotherapy.

### Example 9: Anti-Tumor Efficacy of the Combination Treatment with Anti-PD-1, One Dose of Anti-CTLA4 with Intravenous Delivery of Oncolytic Virus VSV-mlFNb-NIS in Mice Bearing 150 mm³ Average CMT64 Lung Tumors

This example describes the anti-tumor efficacy of a triple combination using the oncolytic virus Vesicular Stomatitis Virus (VSV) encoding IFNb and NIS with anti-PD-1 and one dose of anti-CTLA4 in wild type mice implanted with CMT64 tumors which are resistant to anti-PD-1 treatment. The VSV used in this study is a genetically attenuated virus named VSV-mlFNb-NIS (or mVV1) as it encodes for the mouse interferon beta (IFNb), inserted between the M and G viral genes and for the sodium/iodide symporter (NIS) inserted between the G and L viral genes. The anti-PD-1 antibody used in this study is the clone 29F1.A12 rat IgG2a from Bioxcell, and the anti-CTLA4 antibody used was clone 9D9 in mlgG2a format from Invivogen.

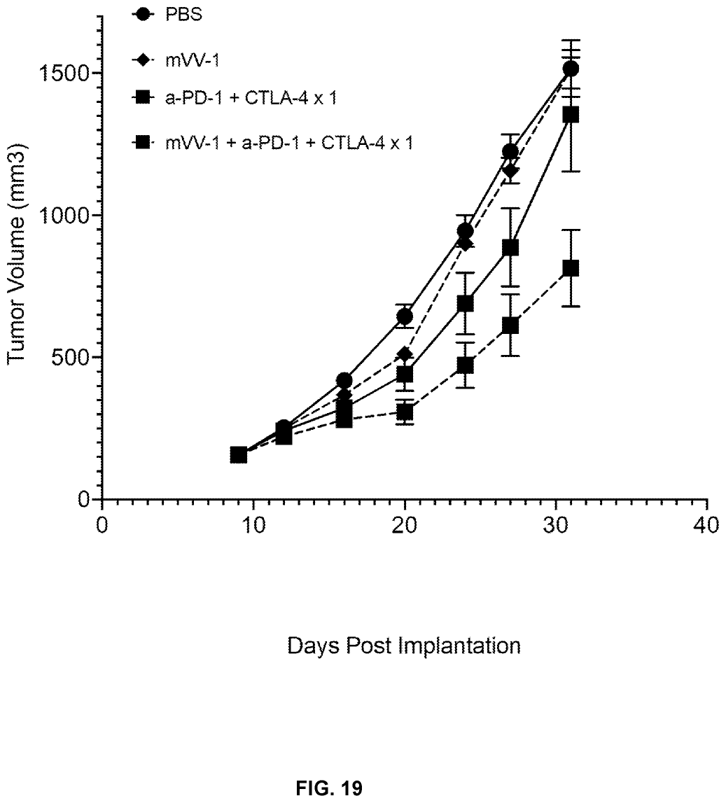

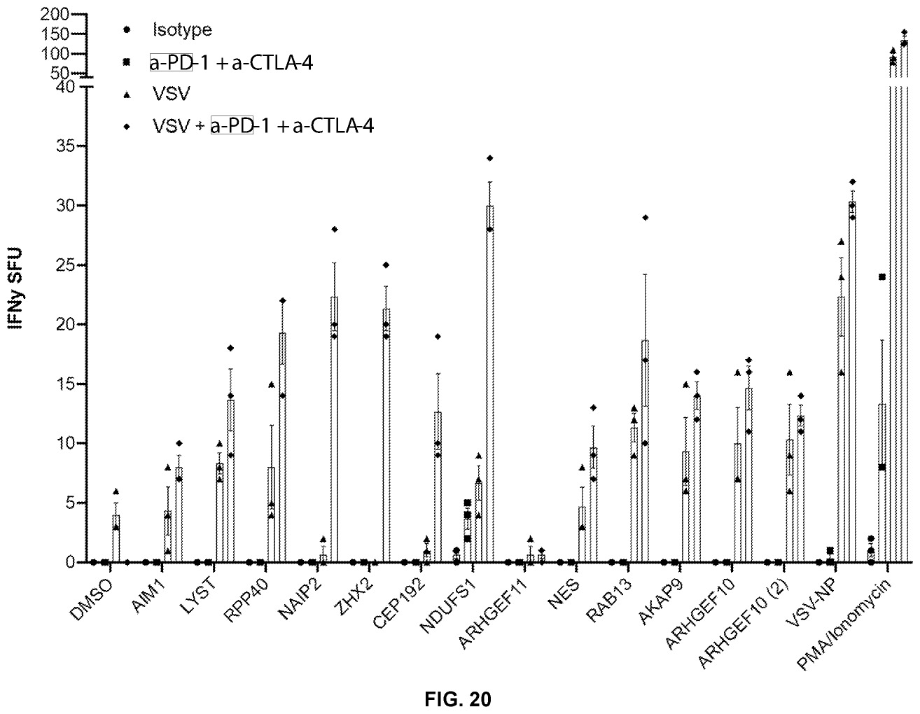

C57BL/6 strain background mice from Jackson Laboratories were implanted subcutaneously with B16F10 cells (5×10⁵ cells/mouse) at day 0. Tumors were measured using a caliper, and tumor volumes were calculated with the formula (L²×W)/2 where L is the smallest size. Mice were randomized evenly into four treatment groups when the average tumor size reached 100 mm³ which was at day 10. Mice received an intravenous injection of 200 μl of mVV1 at either 1×10⁹ TCID50 dose resuspended in PBS or PBS as control, and/or with intraperitoneal injection of 250 μg of either isotype control antibody (mlgG2a and/or rat IgG2a) and/or anti-mouse PD-1 rat IgG2a antibody (29F1.A12) on days 12, 15, 19, and 22 and/or 50 μg of anti-mouse CTLA4-mlgG2a antibody (clone 9D9) as a single dose on day 10. Tumor volumes were monitored by caliper measurements twice per week until the end of the study at day 60. The average of tumor volumes over time for each group shows that one dose of anti-CTLA4 antibodies showed strong tumor growth inhibition in the CMT64 tumor model compared to PBS or anti-PD-1 with anti-CTLA4 (FIG. 19). FIG. 20 shows average spot forming units (SFU) of IFNg released by CD8 TILs harvested from tumors and re-exposed overnight to the indicated tumor antigen or VSV-NP in each treatment group at day 17 after receiving VSV at day 12 along with two doses of anti-PD-1 and a-CTLA4 at day 12 and 14. DMSO and PMA/Ionomycin serve as negative and positive controls respectively multiple post-tumor implantation time points, with treatment days indicated by arrows.

In summary, treatment with a combination of intravenously delivered mVV1 with anti-PD-1 and one dose of anti-mCTLA4 antibodies resulted in surprisingly strong anti-tumor efficacy in the CMT64 anti-PD-1 resistant tumor model, indicating that this triple combination efficacy is applicable to various tumor settings.

TABLE 14
Experimental dosing and treatment protocol for groups of mice

| | | | | | | | Ab 2 | |
|---|---|---|---|---|---|---|---|---|
| | | | | | | | dos- | |
| | Virus | Virus | | Ab 1 | Ab l | Ab 2 | ing | |
| | or PBS | or | | (route | dosing | (route | inter- | n |
| Group | Route | PBS | TCID₅₀ | IP) | interval | IP) | val | # |
| 1 | Intra- | PBS | — | Isotype | D9, 12, | Isotype | D9 | 8 |
| | venous | | | rat | 16, 20, | mouse | | |
| | | | | IgG2a | 24, 27, | IgG2a | | |
| | | | | 250 μg | 31 | 50 μg | | |
| 2 | Intra- | VSV- | 1 × 10⁹ | Isotype | D10, 14, | Isotype | D9 | 8 |
| | venous | mIFNb- | TCID⁵⁰ | rat | 17, 21, | mouse | | |
| | | NIS | | IgG2a | 24, 28 | IgG2a | | |
| | | | | 250 μg | | 50 μg | | |
| 3 | Intra- | PBS | — | a-PD-1 | D10, 14, | a- | D9 | 8 |
| | venous | | | 250 μg | 17, 21, | CTLA4 | | |
| | | | | | 24, 28 | mIgG2a | | |
| | | | | | | 50 μg | | |
| 4 | Intra- | VSV- | 1 × 10⁹ | a-PD-1 | D10, 14, | a- | D9 | 8 |
| | venous | mIFNb- | TCID⁵⁰ | 250 μg | 17, 21, | CTLA4 | | |
| | | NIS | | | 24, 28 | mIgG2a | | |
| | | | | | | 50 μg | | |

TABLE 15
Mean tumor volume, percent survival, and numbers of tumor free
mice in each treatment group from in vivo tumor focused on testing
the triple combination mVV1 intravenous with anti-CTLA4 +
anti-PD-1 antibodies in CMT64 lung adenocarcinoma model.

| | | Tumor Volume, | | | | |
|---|---|---|---|---|---|---|
| | | mm³ mean (±SD) | | Survival, % | | |
| Treatment group (n = 8) | | Day 31 | | Day 31 | Day 41 | Day 59 |
| 1 | PBS | 1516 | (±280) | 100% | 0% | 0% |
| 2 | mVV1 | 1514 | (±193) | 100% | 0% | 0% |
| 5 | a-PD-1 + a-CTLA4 | 1355 | (±568) | 100% | 37.5% | 12.5% |
| 10 | mVV1 + a-PD-1 + a-CTLA4 | 814 | (±381) | 100% | 75% | 12.5% |

As shown in Table 15, mice treated with the triple combination mVV1 intravenous with anti-PD-1 and one dose of anti-CTLA4 antibodies were very efficacious at controlling CMT64 tumor growth.

### Example 10: The Combination Treatment of Anti-PD-1, Anti-CTLA4 with Intra-Venous Delivery of Oncolytic Virus VSV-mlFNb-NIS in Mice Bearing 150 mm³ Average CMT64 Lung Tumors Elicits a Wide Polyclonal Anti-Tumor T Cell Response

This example describes the mechanism of action of a triple combination using the oncolytic virus Vesicular Stomatitis Virus (VSV) encoding IFNb and NIS with anti-PD-1 and one dose of anti-CTLA4 in wild type mice implanted with CMT64 tumors which are resistant to anti-PD-1 treatment. The VSV used in this study is a genetically attenuated virus named VSV-mlFNb-NIS (or mVV1) as it encodes for the mouse interferon beta (IFNb), inserted between the M and G viral genes and for the sodium/iodide symporter (NIS) inserted between the G and L viral genes. The anti-PD-1 antibody used in this study is the clone 29F1.A12 rat IgG2a from Bioxcell, and the anti-CTLA4 antibody used was clone 9D9 in mlgG2a format from Invivogen.

C57BL/6 strain background mice from Jackson Laboratories were implanted subcutaneously with CMT64 cells (5×10⁶ cells/mouse) at day 0. Tumors were measured using a caliper, and tumor volumes were calculated with the formula (L²×W)/2 where L is the smallest size. Mice were randomized evenly into seven treatment groups when the average tumor size reached 100 mm³ which was at day 10. Mice received an intravenous injection of 200 μl of mVV1 at either 1×10⁹ TCID50 dose resuspended in PBS or PBS as control, and/or with intraperitoneal injection of 250 μg of either isotype control antibody (mlgG2a and/or rat IgG2a) and/or anti-mouse PD-1 rat IgG2a antibody (29F1.A12) and/or 10 μg of anti-mouse CTLA4-mlgG2a antibody (clone 9D9) on days 10 and 14. Tumors were harvested at day 17. Purified CD8 TILs and naïve splenocytes were co-incubated at 1:1 ratio by plating 10,000 cells per well and incubated overnight with the respective peptide antigen for an IFNg ELISPOT assay. A large reactivity of the CD8 TILs was detected to be specific to VSV-NP antigen in the groups that received VSV. Notably, many of the tumor neo-antigens were inducing signal in the re-exposed CD8 TILs collected from the group that received VSV, and a very limited response was detected for the groups that were treated with anti-PD-1 and anti-CTLA4 alone. The triple combination VSV with a-PD-1 and a-CTLA4 induced a large polyclonal anti-tumor T cell response compared to the other groups with some neo-antigen reactivities being detected only in the triple combination such as NAIP2 and ZHX2.

This data indicates that the triple combination efficacy is driven by the generation of polyclonal anti-tumor T cells that are functional within the tumor and induce anti-tumor T cell responses.

TABLE 16
Experimental dosing and treatment protocol for groups of mice

| | | | | | | | Ab 2 | |
|---|---|---|---|---|---|---|---|---|
| | Virus | | | | | | dos- | |
| | or | Virus | | Ab 1 | Ab 1 | Ab 2 | ing | |
| | PBS | or | | (route | dosing | (route | inter- | n |
| Group | Route | PBS | TCID₅₀ | IP) | interval | IP) | val | # |
| 1 | Intra- | PBS | — | Isotype | D10, | Isotype | D10, | 8 |
| | venous | | | rat | 14 | mouse | 14 | |
| | | | | IgG2a | | IgG2a | | |
| | | | | 250 μg | | 10 μg | | |
| 2 | Intra- | VSV- | 1 × 10⁹ | Isotype | D10, | Isotype | D10, | 8 |
| | venous | mIFNb- | TCID⁵⁰ | rat | 14 | mouse | 14 | |
| | | NIS | | IgG2a | | IgG2a | | |
| | | | | 250 μg | | 10 μg | | |
| 3 | Intra- | PBS | — | a-PD-1 | D10, | a-CTLA4 | D10, | 8 |
| | venous | | | 250 μg | 14 | mIgG2a | 14 | |
| | | | | | | 10 μg | | |
| 4 | Intra- | VSV- | 1 × 10⁹ | a-PD-1 | D10, | a-CTLA4 | D10, | 8 |
| | venous | mIFNb- | TCID⁵⁰ | 250 μg | 14 | mIgG2a | 14 | |
| | | NIS | | | | 10 μg | | |

TABLE 17
IFNg Elispot data generated from TILs isolated
from CMT64 tumors harvested from mice seven
days post treatment (10,000 TILs: 10,000 splenocytes)

| | | | a-PD-1/ | | | | mW1/a-PD-1/ | |
|---|---|---|---|---|---|---|---|---|
| Elispot | Isotype | | a-CTLA4 | | mW1 | | a-CTLA4 | |
| Antigens | Mean | ±SD | Mean | ±SD | Mean | ±SD | Mean | ±SD |
| DMSO | 0.0 | 0.0 | 0.0 | 0.0 | 4.0 | 1.7 | 0.0 | 0.0 |
| AIM1 | 0.0 | 0.0 | 0.0 | 0.0 | 4.3 | 3.5 | 8.0 | 1.7 |
| LYST | 0.0 | 0.0 | 0.0 | 0.0 | 8.3 | 1.5 | 13.7 | 4.5 |
| RPP40 | 0.0 | 0.0 | 0.0 | 0.0 | 8.0 | 6.1 | 19.3 | 4.6 |
| NAIP2 | 0.0 | 0.0 | 0.0 | 0.0 | 0.7 | 1.2 | 22.3 | 4.9 |
| ZHX2 | 0.0 | 0.0 | 0.0 | 0.0 | 0.0 | 0.0 | 21.3 | 3.2 |
| CEP192 | 0.0 | 0.0 | 0.0 | 0.0 | 1.0 | 1.0 | 12.7 | 5.5 |
| NDUFS1 | 0.7 | 0.6 | 3.7 | 1.5 | 6.7 | 2.5 | 30.0 | 3.5 |
| ARHGEF11 | 0.0 | 0.0 | 0.0 | 0.0 | 0.7 | 1.2 | 0.7 | 0.6 |
| NES | 0.0 | 0.0 | 0.0 | 0.0 | 4.7 | 2.9 | 9.7 | 3.1 |
| RAB13 | 0.0 | 0.0 | 0.0 | 0.0 | 11.3 | 2.1 | 18.7 | 9.6 |
| AKAP9 | 0.0 | 0.0 | 0.0 | 0.0 | 9.3 | 4.9 | 14.0 | 2.0 |
| ARHGEF10 | 0.0 | 0.0 | 0.0 | 0.0 | 10.0 | 5.2 | 14.7 | 3.2 |
| ARHGEF10 | 0.0 | 0.0 | 0.0 | 0.0 | 10.3 | 5.1 | 12.3 | 1.5 |
| (2) | | | | | | | | |
| VSV-NP | 0.0 | 0.0 | 0.3 | 0.6 | 22.3 | 5.7 | 30.3 | 1.5 |
| PMA/ | 1.0 | 1.0 | 13.3 | 9.2 | 93.0 | 15.7 | 135.0 | 17.3 |
| Ionomycin | | | | | | | | |

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The sequence listing of the present application is submitted electronically as a computer readable sequence listing in XML format with a file name of “10975_SeqList_ST26.xml”, a creation date of Nov. 2, 2022, and a size of 30.8 KB. This sequence listing submitted is part of the specification and is hereby incorporated by reference in its entirety. The following sequences have a length that is below the minimum length permitted under ST.26 format: SEQ ID NO: 7 (Ala Ala Ser) and SEQ ID NO: 19 (Ala Ala Ser).

1. A method of treating or inhibiting the growth of a tumor, comprising:
(a) selecting a patient with a cancer; and
(b) administering to the patient in need thereof: (i) a therapeutically effective amount of an oncolytic virus in combination with (ii) a therapeutically effective amount of a programmed death 1 (PD-1) pathway inhibitor comprising an anti-PD-1 antibody or antigen-binding fragment thereof, and (iii) a therapeutically effective amount of a cytotoxic T-lymphocyte antigen-4 (CTLA4) inhibitor comprising an anti-CTLA4 antibody or antigen-binding fragment thereof.

2. The method of claim 1, wherein the oncolytic virus comprises an oncolytic vesiculovirus.

3. The method of claim 1, wherein the oncolytic vesiculovirus comprises an oncolytic vesicular stomatitis virus (VSV).

4. The method of claim 3, wherein the VSV comprises a recombinant VSV.

5. The method of claim 4, wherein the recombinant VSV comprises an M51R substitution.

6. The method of claim 4, wherein the recombinant VSV expresses a cytokine.

7. The method of claim 6, wherein the cytokine comprises an interferon-beta (IFNb).

8. The method of claim 7, wherein a nucleic acid sequence encoding the IFNb is positioned between M and G vial genes.

9. The method of claim 4, wherein the recombinant VSV expresses a sodium/iodide symporter (NIS).

10. The method of claim 9, wherein a nucleic acid sequence encoding the NIS is positioned between G and L viral genes.

11. The method of claim 1, wherein the oncolytic virus is Voyager V1.

12. The method of claim 1, wherein the oncolytic virus, the PD-1 pathway inhibitor, and the CTLA4 inhibitor are administered concurrently to the patient.

13. The method of claim 1, wherein one or more doses of the oncolytic virus are administered sequentially in combination with one or more doses of the PD-1 pathway inhibitor and one or more doses of the CTLA4 inhibitor.

14. The method of claim 13, wherein the one or more doses of the CTLA4 inhibitor comprise a single dose of the CTLA4 inhibitor and wherein administration of the single dose of the CTLA4 inhibitor leads to an anti-tumor efficacy comparable to that with a combination therapy comprising two or more doses of the CTLA4 inhibitor.

15. The method of claim 14, wherein the anti-tumor efficacy is characterized by decrease in mean or average tumor volume, percent survival, numbers of tumor free patients in each treatment group, or a combination thereof.

16. The method of claim 1, wherein the oncolytic virus is administered to the patient as one or more doses of 10⁴-10¹⁴ TCID₅₀, 10⁴-10¹² TCID₅₀, 10⁶-10¹² TCID₅₀, 10⁸-10¹⁴ TCID₅₀, 10⁸-10¹² TCID₅₀ or 10¹⁰-10¹² TCID₅₀.

17. The method of claim 1, wherein the PD-1 pathway inhibitor is administered to the patient in one or more doses of about 0.1 mg/kg to about 20 mg/kg of body weight of the patient.

18. The method of claim 1, wherein the PD-1 pathway inhibitor is administered to the patient in one or more doses of about 1 mg to about 1000 mg.

19. The method of claim 1, wherein the CTLA4 inhibitor is administered to the patient in one or more doses of about 0.1 mg/kg to about 15 mg/kg of body weight of the patient.

20. The method of claim 1, wherein the CTLA4 inhibitor is administered to the patient in a single dose of about 0.1 mg/kg to about 15 mg/kg of body weight of the patient.

21. The method of claim 1, wherein the CTLA4 inhibitor is administered to the patient in one or more doses of about 1 mg to about 600 mg.

22. The method of claim 1, wherein the oncolytic virus is administered intratumorally or intravenously to the patient.

23. The method of claim 1, wherein the PD-1 pathway inhibitor and the CTLA4 inhibitor are administered intravenously, subcutaneously or intraperitoneally to the patient.

24. The method of claim 1, wherein the cancer is selected from adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, lymphoma, melanoma, meningioma, multiple neuroendocrine type I and type II tumors, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumors, prostate cancer, rectal cancer, renal cancer, respiratory cancer, sarcoma, skin cancer, stomach cancer, testicular cancer, thyroid cancer, tracheal cancer, urogenital cancer, and uterine cancer.

25. (canceled)

26. (canceled)

27. The method of claim 1, wherein the anti-PD-1 antibody is selected from cemiplimab, nivolumab, pembrolizumab, pidilizumab, MEDI0608, BI 754091, PF-06801591, spartalizumab, camrelizumab, JNJ-63723283, and MCLA-134.

28. The method of claim 1, wherein the anti-PD-1 antibody or antigen-binding fragment thereof comprises the heavy chain complementarity determining regions (HCDRs) of a heavy chain variable region (HCVR) comprising the amino acid sequence of SEQ ID NO: 1 and the light chain complementarity determining regions (LCDRs) of a light chain variable region (LCVR) comprising the amino acid sequence of SEQ ID NO: 2.

29. The method of claim 1, wherein the anti-PD-1 antibody or antigen-binding fragment thereof comprises three heavy chain complementarity determining regions (HCDRs) (HCDR1, HCDR2, and HCDR3) comprising the respective amino acid sequences of SEQ ID NOs: 3, 4, and 5; and three light chain CDRs (LCDR1, LCDR2, and LCDR3) comprising the respective amino acid sequences of SEQ ID NOs: 6, 7, and 8.

30. The method of claim 1, wherein the anti-PD-1 antibody or antigen-binding fragment thereof comprises a heavy chain variable region (HCVR) comprising the amino acid sequence of SEQ ID NO: 1; and a light chain variable region (LCVR) comprising the amino acid sequence of SEQ ID NO: 2.

31. The method of claim 1, wherein the anti-PD-1 antibody or antigen-binding fragment thereof comprises a heavy chain and light chain sequence pair of SEQ ID NOs: 9 and 10.

32-35. (canceled)

36. The method of claim 1, wherein the anti-CTLA4 antibody is selected from ipilimumab, tremelimumab, and REGN4659.

37. The method of claim 1, wherein the anti-CTLA4 antibody or antigen-binding fragment thereof comprises the heavy chain complementarity determining regions (HCDRs) of a heavy chain variable region (HCVR) comprising the amino acid sequence of SEQ ID NO: 13 and the light chain complementarity determining regions (LCDRs) of a light chain variable region (LCVR) comprising the amino acid sequence of SEQ ID NO: 14.

38. The method of claim 1, wherein the anti-CTLA4 antibody or antigen-binding fragment thereof comprises three heavy chain complementarity determining regions (HCDRs) (HCDR1, HCDR2, and HCDR3) comprising the respective amino acid sequences of SEQ ID NOs: 15, 16, and 17; and three light chain CDRs (LCDR1, LCDR2, and LCDR3) comprising the respective amino acid sequences of SEQ ID NOs: 18, 19, and 20.

39. The method of claim 1, wherein the anti-CTLA4 antibody or antigen-binding fragment thereof comprises a heavy chain variable region (HCVR) comprising the amino acid sequence of SEQ ID NO: 13; and a light chain variable region (LCVR) comprising the amino acid sequence of SEQ ID NO: 14.

40. The method of claim 1, wherein the anti-CTLA4 antibody or antigen-binding fragment thereof comprises a heavy chain and light chain sequence pair of SEQ ID NOs: 21 and 22.

41. The method of claim 1, wherein the treatment produces a therapeutic effect selected from one or more of: delay in tumor growth, reduction in tumor cell number, tumor regression, increase in survival, partial response, and complete response.

42. The method of claim 1, wherein the tumor growth is inhibited by at least 50% as compared to an untreated patient.

43. The method of claim 1, wherein the tumor growth is inhibited by at least 50% as compared to a patient administered the oncolytic virus, the PD-1 pathway inhibitor, or the CTLA4 inhibitor as monotherapy.

44. The method of claim 1, wherein the tumor growth is inhibited by at least 50% as compared to a patient administered any two of the oncolytic virus, the PD-1 pathway inhibitor, and the CTLA4 inhibitor.

45. The method of claim 1, further comprising administering an additional therapeutic agent or therapy to the patient.

46. The method of claim 45, wherein the additional therapeutic agent or therapy is selected from: radiation, surgery, a chemotherapeutic agent, a cancer vaccine, a B7-H3 inhibitor, a B7-H4 inhibitor, a lymphocyte activation gene 3 (LAG3) inhibitor, a T cell immunoglobulin and mucin-domain containing-3 (TIM3) inhibitor, a galectin 9 (GALS) inhibitor, a V-domain immunoglobulin (Ig)-containing suppressor of T-cell activation (VISTA) inhibitor, a Killer-Cell Immunoglobulin-Like Receptor (KIR) inhibitor, a B and T lymphocyte attenuator (BTLA) inhibitor, a T cell immunoreceptor with Ig and ITIM domains (TIGIT) inhibitor, a CD47 inhibitor, an indoleamine-2,3-dioxygenase (IDO) inhibitor, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an antibody to a tumor-specific antigen, Bacillus Calmette-Guerin vaccine, granulocyte-macrophage colony-stimulating factor (GM-CSF), a cytotoxin, an interleukin 6 receptor (IL-6R) inhibitor, an interleukin 4 receptor (IL-4R) inhibitor, an IL-10 inhibitor, IL-2, IL-7, IL-12, IL-21, IL-15, an antibody-drug conjugate, an anti-inflammatory drug, and combinations thereof.

47. (canceled)

48. A kit comprising an oncolytic virus, a PD-1 pathway inhibitor comprising an anti-PD-1 antibody or antigen-binding fragment thereof, and a CTLA4 inhibitor comprising an anti-CTLA4 antibody or antigen-binding fragment thereof, in combination with written instructions for use of a therapeutically effective amount of a combination of the oncolytic virus, the PD-1 pathway inhibitor, and the CTLA4 inhibitor for treating or inhibiting the growth of a tumor of a patient.
